(19) United States
(12) Patent Application Publication
(10) Pub. No.: US 2007/0286863 A1
(43) Pub. Date: Dec. 13, 2007

(54) CMKLR regulation of adipogenesis and adipocyte metabolic function

(76) Inventors: Christopher Sinal, Halifax (CA); Kerry Goralski, Halifax (CA); Tanya McCarthy, Halifax (CA); Luis Zuniga, Stanford, CA (US); Brian A. Zabel, Mountain View, CA (US); Eugene C. Butcher, Portola Valley, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/800,116

(22) Filed: May 4, 2007

Related U.S. Application Data
(60) Provisional application No. 60/801,329, filed on May 17, 2006.

(51) Int. Cl. A61K 39/395; A61K 31/7052; A61P 3/00; G01N 33/53; A61K 38/08

(52) U.S. Cl. 424/143.1; 435/375; 435/7.2; 435/7.92; 514/15; 514/44

(57) ABSTRACT

Methods are provided for regulating adipogenesis and metabolic function in adipocytes by modulating the activity of chemokine-like receptor 1 (CMKLR1). Exemplary agents include those that modulate binding of CMKLR1 to a cognate ligand (e.g., chemerin), those that modulate signaling from CMKLR1, and those that modulate expression of either CMKLR1 or its cognate ligand in target cells. Methods are also provided for screening for agents that find use in regulating fat accumulation in a subject.

### INTRODUCTION

The ever increasing incidence of obesity in children and adults has become a major public health concern. According to a recently published study, approximately 17% of U.S. children and 32% of U.S. adults are clinically obese. Obesity, insulin resistance, elevated blood pressure, elevated plasma glucose, and dyslipidemia comprises the clustering of factors, known as the metabolic syndrome, associated with increased risk of cardiovascular disease and diabetes. As such, there is a need for new therapies that are effective in preventing and treating obesity and its related disease states.

Obesity is an independent risk factor for type II diabetes, cardiovascular diseases and hypertension. While the pathologic mechanisms linking obesity with these co-morbidities are most likely multifactorial, increasing evidence indicates that altered secretion of adipose-derived signaling molecules (adipokines; e.g. adiponectin, leptin, and TNFα) and local inflammatory responses are contributing factors.

White adipose tissue, in addition to serving an important metabolic role, is an active endocrine organ that secretes a number of signaling peptides with diverse biological functions. These signaling molecules, collectively termed adipokines, include: cytokines and related proteins (leptin, tumour necrosis factor α (TNFα), interleukin-6 (IL-6) and chemokine (c-c motif) ligand 2 (CCL2)); proteins of the fibrinolytic cascade (plasminogen activator inhibitor-1 (PAI-1)); complement and complement related proteins (adipsin, acylation stimulating protein (ASP) and adiponectin); vasoactive proteins (renin, angiotensinogen, angiotensin 1 and 11) and other biologically active peptides such as resistin. Adipokines have important autocrine/paracrine roles in regulating adipocyte differentiation and metabolism and local inflammatory responses. Adipokines also have important roles in the regulation of systemic lipid and glucose metabolism through endocrine/systemic actions in the brain, liver and muscle.

The secretion and/or serum level of many adipokines is profoundly affected by the degree of adiposity. This has led to the hypothesis that, in obesity, dysregulation of pro-inflammatory/-diabetic and anti-inflammatory/-diabetic adipokine secretion may serve as a pathogenic link between obesity and type II diabetes and cardiovascular diseases. The identification and characterization of novel adipokines will further our understanding of the endocrine function of white adipose tissue, providing novel molecular targets for the development of treatment strategies for obesity and related diseases.

Human chemokine-like receptor-1 (CMKLR1), a recently de-orphaned G-protein-coupled receptor (GPCR), was initially discovered to be expressed on in vitro monocyte-derived dendritic ligand for CMKLR1, chemerin, was recently discovered [Zabel, et al. J Immunol (2005) 174(1):244-51; Wittamer, V., et al., J Exp Med (2003) 198(7): 977-85; Meder, W., et al., FEBS Left (2003) 555(3):495-9.]. Chemerin has been isolated from ascitic fluid (ovarian carcinoma), inflamed synovial fluid, hemofiltrate, and normal serum [Zabel, et al. J Immunol (2005) 174(1):244-51; Wiftamer, V., et al., J Exp Med (2003) 198(7): 977-85; Meder, W., et al., FEBS Left (2003) 555(3):495-9.].

Chemerin, a heparin binding protein, initially exists in its pro-form, which is 163 amino acids long. Cleavage of pro-chemerin by serine proteases of inflammatory, coagulation, and fibrinolytic cascades, results in the loss of the last 6-11 C-terminal amino acids. This proteolytic cleavage, which can be at a number of different sites in pro-chemerin, generates active chemerin leading to a potent increase in ligand activity. This results in the increased migration of CMKLR1 bearing cells (e.g., macrophages) to chemerin [Wittamer, V., et al., J Immunol (2005) 175(1):487-93, Zabel, B. A., et al., J Biol Chem (2005) 280(41): 34661-6].

### RELEVANT LITERATURE

The use of small molecules to block chemoattractant receptors is reviewed by Baggiolini and Moser (1997) J. Exp. Med. 186:1189-1191.

### SUMMARY OF THE INVENTION

Methods are provided by regulating adipogenesis and/or metabolic function in adipocytes by modulating the activity of chemokine-like receptor 1 (CMKLR1). CMKLR1 signaling is shown to promote the differentiation of preadipocytes into adipocytes, and to modulate the metabolic function of mature adipocytes. In some embodiments of the invention, adipocytic cells, including adipocytes and/or pre-adipocytes, are contacted with an agent that modulates CMKLR1 signaling. The methods find use in the development for therapies and the treatment of disorders of adipose development and function (e.g. lipodystrophy, obesity) as well as the secondary disorders of adipose dysfunction (diabetes, hyperlipidemia, hypertension, cardiovascular disease). In other embodiments of the invention, adipocytic cells are contacted in an in vitro culture system.

The present invention is drawn to methods for regulating adipogenesis and metabolic function in adipocytes. Included in these methods are methods for regulating fat accumulation, where blocking CMKLR1 signaling decreases fat accumulation and adipocyte metabolism, and is useful for the treatment or prevention of obesity in a subject. Administration of a ligand for CMKLR1 signaling, e.g. chemerin or a biologically active analog thereof, increases fat accumulation, and is useful in treating or preventing a wasting condition in a subject (e.g., cancer cachexia or anorexia).

By modulating CMKLR1 activity is meant either potentiating its activity (e.g., using natural or synthetic activation agents) or antagonizing its activity (e.g., using antagonizing or inhibitory agents). Potentiating agents include, but are no limited to, agents that increase or maintain the expression of CMKLR1 (e.g., TGF-β, steroids), agents that activate CMKLR1 activity (e.g., natural or synthetic activating ligands of CMKLR1), agents that activate the intracellular signaling components of the CMKLR1 signaling pathway, and agents that increase expression of a CMKLR1 ligand (e.g., chemerin, resolving E1). Antagonizing agents include, but are not limited to, agents that interfere with the interaction of CMKLR1 with its natural ligands, agents that reduce CMKLR1 expression (e.g., by reducing transcription, by administration of anti-sense oligonucleotides or RNAi, by inducing cell surface receptor desensitization and/or internalization, etc.), agents that reduce expression of endogenous ligands of CMKLR1, and agents that inhibit intracellular signals initiated by the binding of CMKLR1 with its ligands. Potentiating and antagonizing agents of the invention can be any of a variety of types, including but not limited to, monoclonal antibodies, small molecules, chimeric proteins/peptides, bioactive peptides, and interfering RNA.

The present invention is also drawn to methods of screening for agents that can regulate adipogenesis and metabolic function in adipocyte, e.g. for use in a method of modulating fat accumulation in a subject. In general, the screening method is designed to determine whether an agent can modulate (i.e., potentiate or antagonize) CMKLR1 activity in a cell, although cell-free systems may also find use, e.g. to determine the initial binding of a candidate agent to CMKLR1. In certain embodiments, a cell expressing CMKLR1, e.g. cells including, without limitation, preadipocytes, adipocytes, etc. that normally express CMKLR1, or those that are genetically engineered to express CMKLR1, are contacted with a candidate agent and the response to a CMKLR1 ligand(s) is evaluated, e.g., by chemotaxis, receptor/ligand binding, target gene expression, signaling responses, etc. In certain other embodiments, a cell expressing CMKLR1 or a ligand is contacted to an agent and the expression level of CMKLR1 or its ligand is evaluated.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C. Chemerin and CMKLR1 mRNA are highly expressed in adipose tissue. Relative expression of chemerin (A) and CMKLR1 (B) determined in mouse tissues by real time quantitative PCR. The liver served as the reference tissue (expression=1.0) to which all other tissues were compared. (C) The expression of chemerin and CMKLR1 mRNA, adipocyte marker genes (leptin and adiponectin) and stromal vascular marker genes (tnfα and mac-1) were measured in adipocyte and stromal vascular fractions (SVF) of white adipose tissue (WAT). WAT served as the reference fraction (expression=1.0) to which the adipocyte and stromal vascular fractions were compared. *p≦0.05 compared to SVF, ANOVA followed Tukey's HSD test. Each bar represents the mean ±s.e.m. of 3 experiments.

FIGS. 2A-2I. CMKLR1 expression and chemerin expression and secretion increase with adipogenesis. Chemerin (A) and CMKLR1 (B) mRNA were measured by QPCR. Gene expression in undifferentiated (UD) cells served as the reference (expression=1.0) to which the other samples were compared. *p≦0.05 compared to UD, ANOVA followed by Tukey's HSD test. Representative western blot of 24 hr conditioned adipocyte media (C) demonstrated differentiation-dependent detection of a 16 kDa protein that was consistent with the molecular weight of recombinant active mouse chemerin (mchem) (D). 8-day 3T3-L1 adipocytes were fixed in 4% paraformaldehyde and incubated with anti-human CMKLR1 antibody (E) or mouse IgG3 isotype control antibody (F). Detection of CMKLR1 immunoreactivity was performed with a goat anti-mouse IgG3 secondary antibody labeled with Alexa Fluor 488 (green). Cells were counterstained Hoechst 33258 nuclei stain (blue). Twenty-four hr conditioned serum-free media samples from confluent preadipocytes and 3-, 5-, 8- and 13-day differentiated cells-were applied to hCMKLR1-CHO cells at a final dilution of 1:100 and stimulated a specific response (*p≦0.05) compared to control CHO-K1 cells (G). Twenty-four hr serum-free conditioned media samples from confluent preadipocytes or 13-day differentiated adipocytes was tested for the ability to stimulate migration of hCMKLR1-L1.2 and pcDNA-L1.2 pre B lymphoma cells through 5 μm transwell inserts (H). *p≦0.05 compared to L1.2-pcDNA cells, †p<0.05 compared preadipocyte media. The effect of recombinant mouse chemerin (mchem) on the phosphorylation of ERK1 and ERK2 MAPKs in mature adipocytes was determined by western analysis (I). All bar graphs represent the mean ±s.e.m. of at least 3 experiments.

FIGS. 3A-3H. Knockdown of chemerin and CMKLR1 impairs differentiation of 3T3-L1 preadipocytes into adipocytes. Confluent preadipocytes were transduced with crude viral lysates that contained 100-1000 MOI of mchemerin-shRNA (CE 100-CE 1000), mCMKLR1-shRNA (CR 100-CR 1000) or control LacZ-shRNA (LZ 1.00-LZ1000) followed by the standard adipocyte differentiation protocol. UD and VEH represent undifferentiated preadipocytes and non-transduced preadipocytes, respectively. Chemerin, (A) CMKLR1 (B) and PPARy (C) mRNA were measured by QPCR on day-5 after inducing differentiation and expressed relative to VEH control. The relative mRNA expression is shown as the mean ±s.e.m of 4 or 5 samples pooled from two separate experiments (A,C) or 7 samples pooled from three separate experiments (B). Representative western blot analysis of chemerin protein (D) and determination of chemerin activity (E) by the aequorin bioassay in 24 hr conditioned adipocyte media on day-5 post differentiation. Representative Oil red O staining of neutral lipid (F) and phase contrast images (200×) (G) were measured on day-8 after differentiation. Fibroblast cell (white arrow) and Goralski et al. 15 Chemerin: A novel adipokine lipid droplets (black arrow). Adiponectin levels in 24-hr conditioned serum-free media from adipocyte 5-days post-differentiation (H). *p≦0.05 compared to VEH or respective LacZ control, ANOVA followed by Tukey's HSD test.

FIG. 4. Pre differentiation knockdown of chemerin and CMKLR1 alters the expression of adipocyte genes. Confluent preadipocytes were transduced with crude viral lysates that contained 1000 MOI of mchemerin-shRNA (CE 1000), mCMKLR1-shRNA (CR 1000) or control LacZ-shRNA (LZ 1000) followed by the standard adipocyte differentiation protocol. UD and VEH represent undifferentiated preadipocytes and non-transduced preadipocytes respectively. RNA was isolated from the cells on day 5 post-differentiation. For relative quantification of gene expression the VEH group served as the reference (expression=1.0) to which the other groups were compared. The undifferentiated cells are the mean ±s.e.m. of 3 experiments. All other bars represent the mean ±s.e.m of 7 samples pooled from three separate experiments. *p<0.05 compared to VEH, †p<0.05 compared to LZ1000, ‡p<0.05 compared to all other groups one-way ANOVA followed by Tukey's HSD test.

FIGS. 5A-5H. Post-differentiation knockdown of chemerin does not alter adipocyte morphology. Four days after initiating differentiation of preadipocytes, the cells were transduced with crude viral lysates that contained 300-3000 MOI of CE-shRNA, (CE 300-CE 3000) CR-shRNA (CR 300-CR 3000) or control LZ-shRNA (LZ 300-LZ 3000). UD and VEH represent undifferentiated preadipocytes and non-transduced preadipocytes, respectively. Analyses were performed on day-8 post-differentiation. Chemerin, (A) CMKLR1 (B) and PPARγ (C) mRNA were measured by QPCR and expressed relative to VEH control. The relative mRNA expression is shown as the mean ±s.e.m of 6-7 replicates pooled from 3 experiments. Representative western blot detection of chemerin protein (D) and chemerin activity (E) determination in 24 hr conditioned adipocyte media. Representative Oil red O staining of neutral lipid (F) and phase contrast images of adipocytes (200×) (G). Examples of lipid containing adipocytes (black arrows). The effect of CE- and CR-shRNA (1000 MOI) treatment on adiponectin secretion into adipocyte media over a period of 24 hr(H). *p≦0.05 compared to VEH and respective LZ-shRNA controls, one-way ANOVA followed by Tukey's HSD test.

FIG. 6: Post-differentiation shRNA knockdown of chemerin and CMKLR1 differentially alters the expression of adipocyte genes. Confluent preadipocytes were incubated in standard adipocyte differentiation media for 3 days. Cells were transduced with crude viral lysates that contained 1000 MOI of mchemerin-shRNA (CE 1000), mCMKLR1-shRNA (CR 1000) or control LacZ-shRNA (LZ 1000) on the 4th day after starting the differentiation protocol. UD and VEH represent undifferentiated preadipocytes and non-transduced preadipocytes respectively. RNA was isolated from the cells on day 8 post-differentiation. For relative quantification of gene expression the VEH group served as the reference (expression=1.0) to which the other groups were compared. All bars represent the mean ±s.e.m of 3 experiments †p<0.05 compared to LZ 1000 and ‡p<0.05 compared to all other groups, one-way ANOVA followed by Tukey's HSD test.

FIGS. 7A-7B: Effect of chemerin and CMKLR1 knockdown on lipolysis in mature adipocytes. Confluent preadipocytes were differentiated according the standard protocol followed by treatment with 1000 MOI LZ-shRNAi, CE-shRNA and CR-shRNA on day 4 post-differentiation. On day 7 postdifferentiation, cell media was replaced with DMEM+0.1% BSA that contained 0, 0.2 or 1.0 nM recombinant mouse chemerin. Twenty-four hours later, fresh media (DMEM+0.1% BSA) without or with 2 μM isoproterenol (ISO) (A) or 100 μM IBMX (B) was added. Lipolysis was determined by measuring media glycerol content 2 hr later. Each bar is the mean ±s.e.m of 3 experiments. ‡p<0.05 compared to basal lipolysis in control, LZ1000 and CR1000 treated groups regardless of chemerin treatment, *p<0.05 compared to the within group isoproterenol stimulated lipolysis in the absence of chemerin and †p<0.05 compared to IBMX stimulated lipolysis in control and LZ1000 treatment groups, three-way ANOVA, followed by Tukey's HSD test.

FIGS. 8A-8F: Human Chemerin and CMKLR1 are highly expressed in human subcutaneous adipose tissue and primary adipocytes. The relative expression of human Chemerin (A) and CMKLR1 (B) mRNA were determined in liver, subcutaneous white adipose tissue (SC WAT) from 2 human donors, ovarian carcinoma cells (OVA), Hepatoma (HEPG2) cells, immature dendritic cells (DC) and placenta by real-time quantitative PCR. The liver served as the reference tissue (expression=1.0) to which all other tissues or cells were compared. Expression of Chemerin (C), CMKLR1 (D) and PPARγ (E) genes in 15-day differentiated human subcutaneous adipocytes relative to confluent preadipocytes expressed as mean ±s.e.m. of 3 experiments. *p<0.05 compared to preadipocytes. The effect of recombinant human chemerin (hchem) on the phosphorylation of ERK1 and ERK2 MAP kinases in primary human adipocytes was determined by western analysis (F).

FIG. 9: The role of chemerin and CMKLR1 in adipose tissue biology. Chemerin and the cognate receptor CMKLR1 are highly expressed in adipocytes (1). Chemerin is secreted either in the active form, or rapidly activated by extracellular proteolytic processing (2). Our findings demonstrate that chemerin and CMKLR1 are required for optimal differentiation (3) and that both genes have modulatory effects on the expression of adipocyte genes involved in lipid and glucose metabolism (4). Furthermore, secreted chemerin may have a role in mediating recruitment of CMKLR1-expressing cells (e.g. macrophages) to adipose tissue. The activation of intracellular ERK1/2 signaling (6) upon treatment of adipocytes with chemerin provides evidence for autocrine/paracrine action and is consistent with activation of CMKLR1.

FIG. 10. Development of a polyclonal antibody against mouse chemerin. Panels A to D, COS7 cells (250,000/well) plated on 12-well plates were transiently transfected with 500 ng of mchemerin-pFlag-CMV-5a plasmid using GeneJuice transfection reagent (Novagen) according to the manufacturers' instructions. Total cell lysates (24 hr) (A, B) or culture media (0, 8, 24 and 30 hr) (C, D) were separated on a 15% polyacrylamide gel, transferred to nitrocellulose membrane and probed with chemerin antiserum (1:200 dilution) or Flag antibody (1:1000 dilution). The chemerin antiserum (A, C) and flag antibody (B, D) detected the 18 kDa chemerin-flag construct in total cell lysates or media. The chemerin antiserum (A) but not the flag antibody (B) detected the 16 kDa recombinant mouse chemerin. Panels (A) and (B); lane 1, recombinant mouse chemerin (25 ng); lane 2, 3, 4 and 5, mchemerin-Flag-CMV-5a transfected cells; lane 6, Flag positive control; lane 7, pFlag-CMV-5a transfected cells. Panels (C) and (D); lane 1, 30 hr media; lane 2, 24 hr media; lane 3, 8 hr media; lane 4, 0 hr media; lane 5, chemerin-pFlag-CMV transfected COS7 cell lysate. These results strongly support the detection of mouse chemerin with this antiserum.

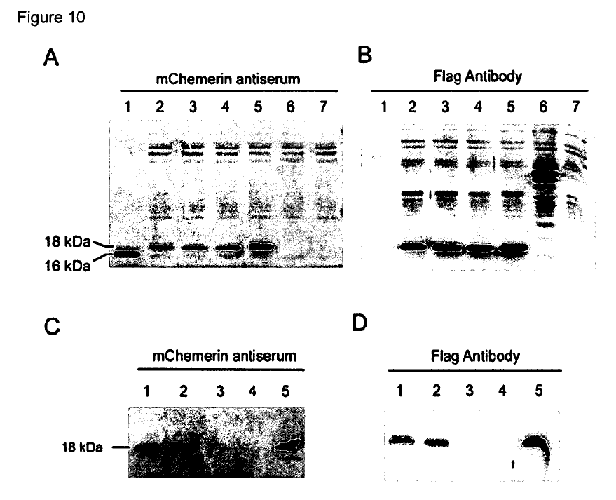

FIG. 11. Validation of the 3T3-L1 adipocyte model, chemerin bioassay and chemerin migration assays. 3T3-L1 preadipocytes (A) were stimulated to undergo differentiation into lipid containing adipocytes (B) by treatment with insulin (850 nM), dexamethasone (250 nM) and IBMX (100 μM) for 3 days. Adipocyte differentiation (C) is characterized by rounded cell morphology and progressive lipid accumulation as shown by neutral lipid staining with Oil Red O. The adipocyte differentiation marker genes PPARγ (D) and leptin (E) were measured by QPCR and increased in expression during adipogenesis. Gene expression in undifferentiated (UD) cells served as the reference (expression=1) to which the other samples were compared. Each bar represents the mean ±s.e.m. of 3 samples. *p≦0.05 compared to UD. Mouse chemerin (□) stimulates the human CMKLR1-G_{α16}-aequorin reporter assay with a similar dose response profile as compared to human chemerin (▪) (F). Mouse (∘) or human (●) chemerin do not stimulate a response in the control G_{α16}-aequorin-CHO cells. The calculated Kₘ for mouse chemerin (119±29 pM) is approximately 2.5-fold higher than the Kmfor human recombinant chemerin (48±12 pM). Twenty-four hr conditioned serum-free media from 3T3-L1 cells at different stages of differentiation were diluted 1:20, 1:100, 1:200, 1:400 and 1:2000 into serum free and phenol red free DMEM/F12. The diluted samples were tested for the activation of the chemerin-aequorin reporter gene assay (G). With increased time after differentiation the media response curve was shifted left. A dilution of 1:100 produced a response that was in the linear range for all samples. In a dose dependent fashion, mouse chemerin stimulates the migration of hCMKLR1-L1.2 cells through 5 μm transwell inserts and compared to pcDNA-transfected control cells (H).

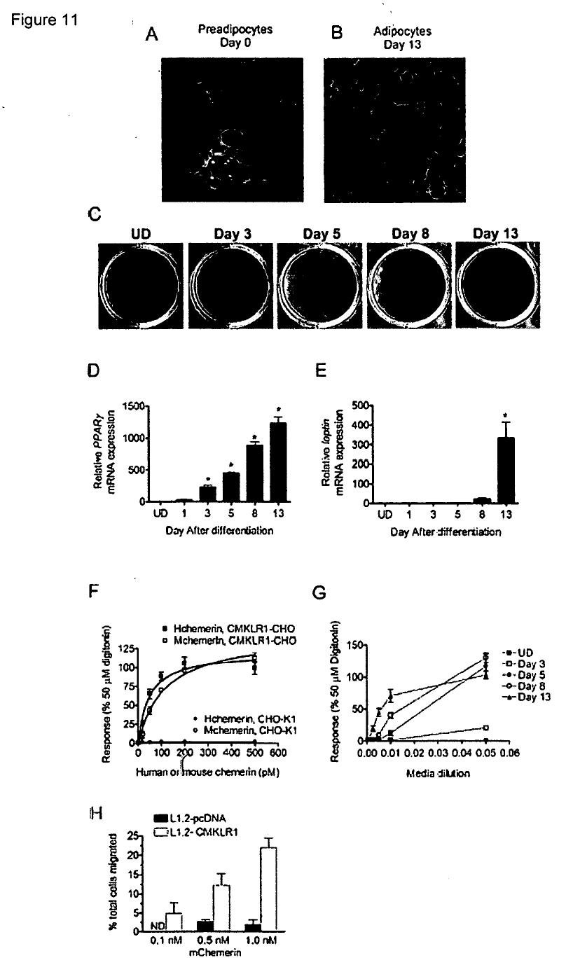

FIG. 12. Chemerin and CMKLR1 shRNA treatment produces time-dependent changes in adipocyte morphology. Confluent preadipocytes were transduced with crude viral lysates that contained 1000 MOI of mchemerin-shRNA (CE 1000), mCMKLR1-shRNA (CR 1000) or control LacZ-shRNA (LZ 1000) followed by the standard adipocyte differentiation protocol. UD and VEH represent undifferentiated preadipocytes and non-transduced preadipocytes, respectively. Phase contrast images (200×) of unstained living cells were measured on day 3, 4, 6 and 8 after initiating adipocyte differentiation.

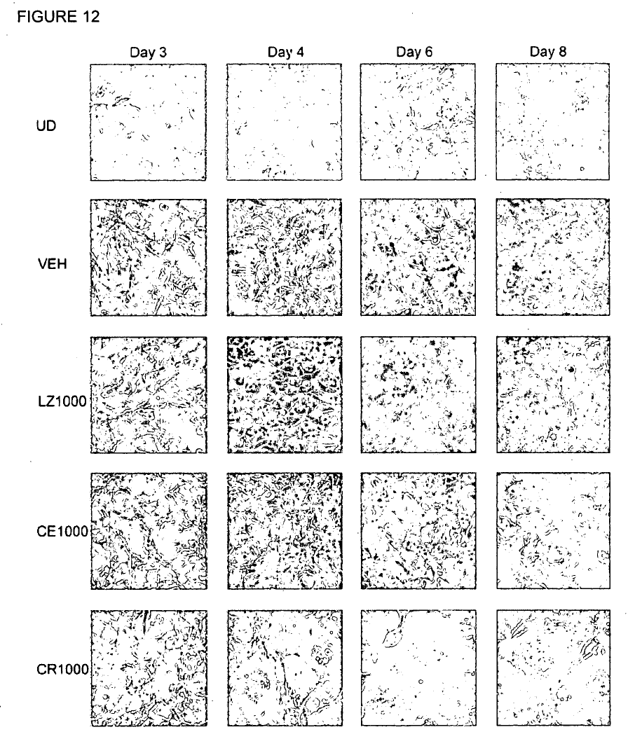

FIG. 13. Epididymal white adipose tissue (WAT) was collected from obese (ob/ob) leptin deficient mice and wild-type (WT) litter-mate controls that express normal amounts of leptin. Mice were 12-weeks of age. White adipose tissue was digested by collagenase and separated into adipocyte and stromal vascular fractions (SVF). RNA was isolated from each fraction and chemerin and CMKLR1 (chemerinR) mRNA expression was measured by quantitative PCR. Left panel, chemerin expression was lower (60-70%) in the SVF fraction compared to the adipocyte fraction and whole adipose tissue, †P<0.05, ANOVA. There was no difference in WAT and adipocyte expression of chemerin in ob/ob mice versus controls. Chemerin expression was about 50% lower in the SVF from ob/ob mice but not significantly different from the WT SVF. Right panel, CMKLR1 expression was lower in WAT and the adipocyte fraction of ob/ob mice compared to WT controls, *P<0.05 ANOVA. CMKLR1 expression was lower in the SVF compared to WAT and adipocyte fractions in WT mice †P<0.05, ANOVA. There was no difference In SVF expression of CMKLR1 in the ob/ob mice compared to the WT controls. These results suggest that CMKLR1 might be regulated by leptin and/or by the obese state. The loss in CMKLR1 expression in obese WAT was restricted to adipocytes.

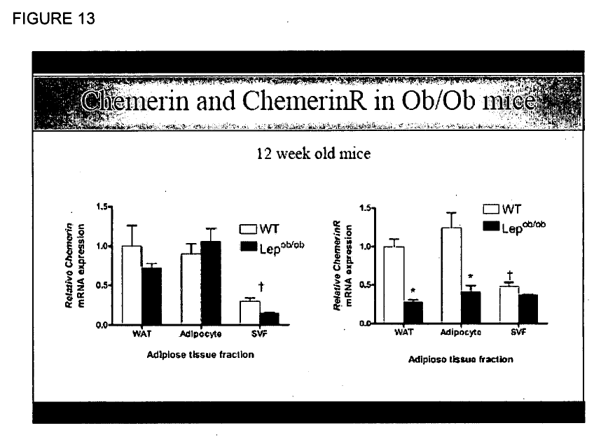

### DESCRIPTION OF THE SPECIFIC EMBODIMENTS

As summarized above, the present invention is drawn to methods for regulating adipogenesis and/or metabolic function in adipocytes by modulating the activity of chemokine-like receptor 1 (CMKLR1). Cells of interest are contacted with an agent that modulates (i.e., potentiates or antagonizes) the natural activity of chemokine-like receptor 1 (CMKLR1) and/or a CMKLR1 ligand (e.g., chemerin or other endogenous CMKLR1 ligands). As such, the methods of the invention find use in treating disorders of adipose development and function (e.g. lipodystrophy, obesity) as well as the secondary disorders of adipose dysfunction (diabetes, hyperlipidemia, hypertension, cardiovascular disease); as well as treating or preventing a wasting condition in a subject. Methods of screening for agents that regulate adipogenesis and/or metabolic function in adipocytes are also provided.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The sequence of CMKLR1 may be found in Genbank, accession number Y14838, and is described by Samson et al., (1998) Eur J Immunol. 28(5):1689-700. The sequence of a CMKLR1 ligand, mammalian chemerin, may be found in Genbank, accession number NM_{—}002889, which encodes the polypeptide:

MRRLLIPLALWLGAVGVGVAELTEAQRRGLQVALEEFHKHPPVQWAFQETSVESAVDTP
FPAGIFVRLEFKLQQTSCRKRDWKKPECKVRPNGRKRKCLACIKLGSEDKVLGRLVHCPI
ETQVLREAEEHQETQCLRVQRAGEDPHSFYFPGQFAFSKALPRS.

The active form of chemerin is a cleavage product (see Zabel et al. (2005) JBC 41, 34661-34666 herein specifically incorporated by reference for the teaching of chemerin processing and fragments. At least three endogenously active human chemerin isoforms have been isolated, all with different carboxyl-terminal truncations, and as used herein the term “chemerin” may refer to any one or a combination of these active forms.

Adipocytes are the cells of adipose tissue. There are two types of adipose tissue: white adipose tissue (WAT) and brown adipose tissue (BAT). White fat cells contain a large lipid droplet surrounded by a ring of cytoplasm. The nucleus is flattened and located on the periphery. The fat stored is in a semi-liquid state, and is composed primarily of triglycerides and cholesterol ester. White fat cells secrete a variety of adipokines, including, without limitation, resistin, adiponectin and leptin. Preadipocytes are fibroblastic cells that can be stimulated to form adipocytes, and are located within the mesenchymal cell lineage.

Fat cell differentiation begins with the expression of two families of gene regulatory proteins: the C/EBP (CCAAT/enhancer binding protein) family and the PPAR (peroxisome proliferator-activated receptor) family, especially PPARγ. The C/EBP and PPARγ proteins drive and maintain one another's expression, through various cross-regulatory and autoregulatory control loops. They work together to control the expression of the other genes characteristic of adipocytes.

The production of enzymes for import of fatty acids and glucose and for fat synthesis leads to an accumulation of fat droplets, consisting mainly of triacylglycerol. These then coalesce and enlarge until the cell is hugely distended (up to 120 μm in diameter), with only a thin rim of cytoplasm around the mass of lipid. Lipases are also made in the fat cell, giving it the capacity to reverse the process of lipid accumulation, by breaking down the triacylglycerols into fatty acids that can be secreted for consumption by other cells. The fat cell can change its volume by a factor of a thousand as it accumulates and releases lipid.

Adipogenesis has been studied extensively in vitro using a number of preadipocyte cell lines, including 3T3-L1 cells. When cultured in defined media, 3T3-L1 cells deposit triglyceride in cytoplasmic lipid droplets and express genes that are also expressed in adipocytes in vivo. Key regulatory genes that are necessary and/or sufficient for the transition of preadipocytes into adipocytes include C/EBP (CCAAT/enhancer binding protein) family and the PPAR (peroxisome proliferator-activated receptor) family, especially PPARγ. Studies of these transcription factors have suggested that adipogenesis is the result of a temporally ordered pattern of 3-5 distinct phases of gene expression.

Gene expression changes during adipocyte differentiation in 3T3-L1 cells include the transcription factors C/EBPalpha, PPARgamma 2, SREBP-1, C/EBPbeta, C/EBPdelta, CHOP-10, AEBP1, COUP-TF. A large group of genes is repressed during in vitro adipogenesis, including cell cycle-related genes. 3T3-L1 cells, after the addition of growth factors at confluence, go through a clonal expansion phase that consists of 1-2 rounds of cell division prior to terminal differentiation. Prior to the addition of adipogenic factors, cells are growth-arrested at confluence. After the addition of adipocyte-inducing factors that serves to induce cell division, this cluster of genes returns to preconfluent (dividing cell) expression levels. After this time, at which cells are known to have entered terminal differentiation, this cluster of genes is repressed permanently.

Formulations and Methods of Use

The subject invention provides methods for regulating adipogenesis and/or metabolic function in adipocytes by contacting adipocytic cells with an effective amount of a CMKLR1 modulatory agent. In some embodiments of the invention, the regulating is in an in vitro cell model for adipogenesis. Such in vitro models include cell lines, e.g. 3T3-L1 cells, which undergo adipogenesis in vitro. Other models utilize primary cells, e.g. adipocytes, pre-adipocytes, and stem cells such as mesenchymal stem cells. Such cells may be derived from any suitable mammalian source, e.g. mice, rats, primates including humans, etc. Alternatively, cells in culture may be engineered to express CMKLR1. Analysis of cells may include the histological development of adipocytic morphology, e.g. Oil Red O staining of neutral lipid, etc.; changes in protein state, e.g. ERK1/2 phosphorylation; changes in gene expression, e.g. C/EBP; PPARy, perilipin, glucose transporter-4, (GLUT4), adiponectin hormone sensitive lipase, glycerolphosphate acteyltransferase (GPAT), diacylglycerol3-phosphate acteyltransferase-2 (DGAT2), TNFα, fatty acid synthase (FAS), etc. As shown herein, blocking activation of CMKLR1 in pre-adipocytes decreases adipogenesis. Blocking activation of CMKLR1 in mature adipocytes down-regulates their metabolic function.

In other embodiments of the invention, CMKLR1 activation is modulated in vivo, by contacting a subject with an effective amount of a CMKLR1 modulatory agent.

Modulatory agents include those that potentiate or antagonize the activity of CMKLR1 activity. CMKLR1 modulatory agents can be any of a variety of types, including, but not limited to, monoclonal antibodies, small molecules, chimeric proteins/peptides, bioactive peptides, lipids, cytokines, derivatives thereof and/or interfering RNA.

In certain embodiments, a CMKLR1 modulatory agent is derived from a naturally occurring ligand. For example, a nonamer peptide sequence from chemerin is an activating ligand for CMKLR1 [i.e., amino acids 149-157 (Wittamer JBC 2004), having the amino acid sequence: (NH₂)YFPGQFAFS(COOH)]. In addition, targeted or random mutagenesis techniques can be used to generate mutant forms of a natural ligand for CMKLR1 (e.g., chemerin mutants) and tested for their modulatory activity. Such methods are well known in the art.

In other embodiments, a CMKLR1 modulatory agent blocks CMKLR1 or chemerin expression, e.g. by introduction of an anti-sense molecule or RNAi that acts to decrease expression. Alternatively, an agent such an antibody that blocks the interaction between CMKLR1 and a natural ligand is administered.

Mammalian species that may benefit from regulating adipogenesis and adipocyte metabolism include, but are not limited to, canines; bovines; ovines; etc. and primates, particularly humans. Animal models, particularly small mammals, e.g. murine, lagomorpha, etc. may be used for experimental investigations. Animal models of interest include those related to obesity or a wasting syndrome.

The CMKLR1 modulatory agent may be delivered in any number of ways, including systemically (e.g., orally in the form of a pill or elixir) or delivered directly to a site of interest. The desired administration method may provide for a localized concentration by use of a sustained release formulation. Preliminary doses can be determined according to animal tests, and the scaling of dosages for human administration can be performed according to art-accepted practices.

A variety of sustained release formulations are known and used in the art. For example, biodegradable or bioerodible implants may be used. The implants may be particles, sheets, patches, plaques, fibers, microcapsules and the like and may be of any size or shape compatible with the selected site of insertion. Characteristics of the polymers will include biodegradability at the site of implantation, compatibility with the agent of interest, ease of encapsulation, the half-life in the physiological environment, water solubility, and the like.

Another approach involves the use of an implantable drug delivery device. Examples of such implantable drug delivery devices include implantable diffusion systems (see, e.g., subdermal implants (such as NORPLANT^{ff●}) and other such systems, see, e.g., U.S. Pat. Nos. 5,756,115; 5,429,634; 5,843,069). These implants generally operate by simple diffusion, e.g., the active agent diffuses through a polymeric material at a rate that is controlled by the characteristics of the active agent formulation and the polymeric material. Alternatively, the implant may be based upon an osmotically-driven device to accomplish controlled drug delivery (see, e.g., U.S. Pat. Nos. 3,987,790, 4,865,845; 5,057,318; 5,059,423; 5,112,614; 5,137,727; 5,234,692; 5,234,693; and 5,728,396). These osmotic pumps generally operate by imbibing fluid from the outside environment and releasing corresponding amounts of the therapeutic agent.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure, e.g., intravascular (intravenous, intraarterial, intracapillary) administration. The effective amount of a therapeutic composition to be given to a particular patient will depend on a variety of factors, several of which will be different from patient to patient. A competent clinician will be able to determine an effective amount of a therapeutic agent. The compositions can be administered to the subject in a series of more than one administration. For therapeutic compositions, regular periodic administration (e.g., every 2-3 days) will sometimes be required, or may be desirable to reduce toxicity. For therapeutic compositions that will be utilized in repeated-dose regimens, antibody moieties that do not provoke immune responses are preferred.

The compositions of the invention can be administered in conjunction with other active compounds (e.g, appetite suppressants) as are deemed safe and effective in treating a subject to reduce fat accumulation. Such additional active compounds may be provided in a co-formulation with the CMKLR1 inhibitory agents or as independent compositions.

Those of skill will readily appreciate that dose levels can vary as a function of the specific compound, the severity of the symptoms and the susceptibility of the subject to side effects. Some of the specific complexes are more potent than others. Preferred dosages fora given agent are readily determinable by those of skill in the art by a variety of means. A preferred means is to measure the physiological potency of a given compound.

### Methods of Screening for CMKLR1 Antagonists

Agents that can regulate adipogenesis and adipocyte metabolism in a subject can be identified by detecting the ability of an agent to modulate (i.e., potentiate or antagonize) the activity of CMKLR1. Potentiating agents include, but are not limited to, agents that increase or maintain the expression of CMKLR1 (e.g., TGF-β, steroids), agents that activate CMKLR1 activity (e.g., natural or synthetic activating ligands of CMKLR1), agents that activate the intracellular signaling components of the CMKLR1 signaling pathway, and agents that increase expression of a CMKLR1 ligand (e.g., chemerin, resolving E1). Antagonizing agents include, but are not limited to, agents that interfere with the interaction of CMKLR1 with its natural ligands, agents that reduce CMKLR1 expression (e.g., by reducing transcription or by inducing cell surface receptor desensitization, internalization and/or degradation), agents that reduce expression of endogenous ligands of CMKLR11, and agents that inhibit intracellular signals initiated by the binding of CMKLR1 with its ligands.

In certain embodiments, agents that can reduce fat accumulation in a subject can be identified by detecting the ability of an agent to interfere with the interaction of CMKLR1 with its cognate ligand (e.g., chemerin). For example, a screening assay may be used that evaluates the ability of an agent to bind specifically to CMKLR1 (or its ligand) and prevent receptor:ligand interaction. Assays to determine affinity and specificity of binding are known in the art, including competitive and non-competitive assays. Assays of interest include ELISA, RIA, flow cytometry, etc. Binding assays may use purified or semi-purified protein, or alternatively may use primary cells or immortalized cell lines that express CMKLR1. In certain of these embodiments, the cells are transfected with an expression construct for CMKLR1. As an example of a binding assay, CMKLR1 is inserted into a membrane, e.g. whole cells, or membranes coating a substrate, e.g. microtiter plate, magnetic beads, etc. The candidate agent and soluble, labeled ligand (e.g., chemerin) are added to the cells, and the unbound components are then washed off. The ability of the agent to compete with the labeled ligand for receptor binding is determined by quantitation of bound, labeled ligand. Confirmation that the blocking agent does not cross-react with other chemoattractant receptors may be performed with a similar assay.

CMKLR1 protein sequences are used in screening of candidate compounds (including antibodies, peptides, lipids, small organic molecules, etc.) for the ability to bind to and modulate CMKLR1 activity. Agents that inhibit or reduce CMKLR1 activity are of interest as therapeutic agents for decreasing fat accumulation in a subject whereas agents that activate CMKLR1 activity are of interest as therapeutic agents for increasing fat accumulation in a subject. Such compound screening may be performed using an in vitro model, a genetically altered cell or animal, or purified protein corresponding to chemerin-like chemoattractant polypeptides or a fragment(s) thereof. One can identify ligands or substrates that bind to and modulate the action of the encoded polypeptide.

Polypeptides useful in screening include those encoded by the CMKLR1 gene, as well as nucleic acids that, by virtue of the degeneracy of the genetic code, are not identical in sequence to the disclosed nucleic acids, and variants thereof.

CMKLR1 ligands (e.g., chemerin or resolving E1) are used in screening of candidate compounds (including antibodies, peptides, lipids, small organic molecules, etc.) for the ability to bind to and modulate the ligands ability to activate CMKLR1. Agents that inhibit or reduce the ability of a CMKLR1 ligand to activate CMKLR1 are of interest as therapeutic agents for decreasing fat accumulation in a subject whereas agents that increase the ability of a CMKLR1 ligand to activate CMKLR1 activity are of interest as therapeutic agents for increasing fat accumulation in a subject. Such compound screening may be performed using an in vitro model, a genetically altered cell or animal, or purified protein corresponding to chemerin-like chemoattractant polypeptides or a fragment(s) thereof. One can identify ligands or substrates that bind to and modulate the action of the encoded polypeptide.

Polypeptides useful in screening include those encoded by a CMKLR1 ligand gene (e.g., chemerin), as well as nucleic acids that, by virtue of the degeneracy of the genetic code, are not identical in sequence to the disclosed nucleic acids, and variants thereof.

Transgenic animals or cells derived therefrom are also used in compound screening. Transgenic animals may be made through homologous recombination, where the normal locus corresponding to chemerin-like chemoattractant is altered. Alternatively, a nucleic acid construct is randomly integrated into the genome. Vectors for stable integration include plasmids, retroviruses and other animal viruses, YACs, and the like. A series of small deletions and/or substitutions may be made in the coding sequence to determine the role of different exons in receptor binding, signal transduction, etc. Specific constructs of interest include antisense sequences that block expression of the targeted gene and expression of dominant negative mutations. A detectable marker, such as lac Z or GFP, may be introduced into the locus of interest, where up-regulation of expression will result in an easily detected change in phenotype. One may also provide for expression of the target gene or variants thereof in cells or tissues where it is not normally expressed or at abnormal times of development, for example by overexpressing in neural cells. By providing expression of the target protein in cells in which it is not normally produced, one can induce changes in cell behavior.

Compound screening identifies agents that modulate CMKLR1 activity or function. Of particular interest are screening assays for agents that have a low toxicity for normal human cells. A wide variety of assays may be used for this purpose, including labeled in vitro protein-protein binding assays, electrophoretic mobility shift assays, immunoassays for protein binding, and the like. Screening for the activity of G-protein coupled receptors (or GPCRs, of which CMKLR1 is a member) is well known in the art, and includes assays for measuring any of a number of detectible steps, including but not limited to: stimulation of GDP for GTP exchange on a G protein; alteration of adenylate cyclase activity; protein kinase C modulation; phosphatidylinositol breakdown (generating second messengers diacylglycerol, and inositol triphosphate); intracellular calcium flux; activation of MAP kinases; modulation of tyrosine kinases; modulation of gene or reporter gene activity, integrin activation, or chemotaxis inhibition. A detectable step in a signaling cascade is considered modulated if the measurable activity is altered by 10% or more above or below a baseline or control level. The baseline or control level can be the activity in the substantial absence of an activator (e.g., a ligand) or the activity in the presence of a known amount of an activator. The measurable activity can be measured directly, as in, for example, measurement of cAMP or diacylglycerol levels. Alternatively, the measurable activity can be measured indirectly, as in, for example, a reporter gene assay. Knowledge of the 3-dimensional structure of the encoded protein (e.g., CMKLR1 or a ligand, e.g. chemerin), derived from crystallization of purified recombinant protein, could lead to the rational design of small drugs that specifically inhibit activity. These drugs may be directed at specific domains and sites.

Assays of interest include cell based assays, and may be competitive or non-competitive assays. For example, a candidate agent may be added to a culture of pre-adipocytic or other adipocytic cells in the absence or presence of a CMKLR1 ligand, such as chemerin, and the inhibition or potentiation of adipogenesis or adipocyte metabolism monitored. Alternatively, a candidate agent may be compared for activity with a native ligand, or other known modulating agent. Analysis of cells may include the histological development of adipocytic morphology, e.g. Oil Red O staining of neutral lipid, etc.; changes in protein state, e.g. ERK1/2 phosphorylation; changes in gene expression, e.g. C/EBP; PPARy, perilipin, glucose transporter-4, (GLUT4), adiponectin hormone sensitive lipase, glycerolphosphate acteyltransferase (GPAT), diacylglycerol3-phosphate acteyltransferase-2 (DGAT2), TNFα, fatty acid synthase (FAS), etc.

The term “agent” as used herein describes any molecule, e.g. protein or pharmaceutical, with the capability of modulating the physiological function of CMKLR1 or its ligand. Generally a plurality of assay mixtures is run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, lipids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally. Cho et al., Pac. Symp. Biocompat. 305-16, 1998); Sun et al., J. Comput. Aided Mol. Des. 12:597-604, 1998); each incorporated herein by reference in their entirety). For example, libraries of phosphatase inhibitors can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25, 1994; Burbaum et al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

A “combinatorial library” is a collection of compounds in which the compounds comprising the collection are composed of one or more types of subunits. Methods of making combinatorial libraries are known in the art, and include the following: U.S. Pat. Nos. 5,958,792; 5,807,683; 6,004,617; 6,077,954; which are incorporated by reference herein. The subunits can be selected from natural or unnatural moieties. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is generated in a systematic way. Any method of systematically generating a collection of compounds differing from each other in one or more of the ways set forth above is a combinatorial library.

A combinatorial library can be synthesized on a solid support from one or more solid phase-bound resin starting materials. The library can contain five (5) or more, preferably ten (10) or more, organic molecules that are different from each other. Each of the different molecules is present in a detectable amount. The actual amounts of each different molecule needed can vary due to the actual procedures used and can change as the technologies for isolation, detection and analysis advance. When the molecules are present in substantially equal molar amounts, an amount of 100 picomoles or more can be detected. Preferred libraries comprise substantially equal molar amounts of each desired reaction product and do not include relatively large or small amounts of any given molecules so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin. After attachment of the starting compound, substituents are attached to the starting compound. Substituents are added to the starting compound, and can be varied by providing a mixture of reactants comprising the substituents. Examples of suitable substituents include, but are not limited to, hydrocarbon substituents, e.g. aliphatic, alicyclic substituents, aromatic, aliphatic and alicyclic-substituted aromatic nuclei, and the like, as well as cyclic substituents; substituted hydrocarbon substituents, that is, those substituents containing nonhydrocarbon radicals which do not alter the predominantly hydrocarbon substituent (e.g., halo (especially chloro and fluoro), alkoxy, mercapto, alkylmercapto, nitro, nitroso, sulfoxy, and the like); and hetero substituents, that is, substituents which, while having predominantly hydrocarbyl character, contain other than carbon atoms. Suitable heteroatoms include, for example, sulfur, oxygen, nitrogen, and such substituents as pyridyl, furanyl, thiophenyl, imidazolyl, and the like. Heteroatoms, and typically no more than one, can be present for each carbon atom in the hydrocarbon-based substituents. Alternatively, there can be no such radicals or heteroatoms in the hydrocarbon-based substituent and, therefore, the substituent can be purely hydrocarbon.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 3 hours will be sufficient.

Preliminary screens can be conducted by screening for compounds capable of binding to CMKLR1 or its ligand; compounds so identified are possible modulators. Compounds capable of binding to CMKLR1 are inhibitors if they do not activate the receptor and activators if they do. The binding assays usually involve contacting CMKLR1 or its ligand with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots (see, e.g., Bennet, J. P. and Yamamura, H. I. (1985) “Neurotransmifter, Hormone or Drug Receptor Binding Methods,” in Neurotransmitter Receptor Binding (Yamamura, H. I., et al., eds.), pp. 61-89.

Certain screening methods involve screening for a compound that modulates the expression of CMKLR1 or its ligand. Such methods generally involve conducting cell-based assays in which test compounds are contacted with one or more cells expressing CMKLR1 or its ligand and then detecting a modulation in expression (e.g., at the mRNA and/or protein level). In certain screening methods, a target cell has a reporter gene (e.g., GFP) under the control of the CMKLR1 promoter (or promoter of its ligand). The level of expression can be compared to a baseline value. The baseline value can be a value for a control sample or a statistical value that is representative of expression levels for a control population. Expression levels can also be determined for cells that do not express the CMKLR1 or its ligand, as a negative control. Such cells generally are otherwise substantially genetically the same as the test cells. Various controls can be conducted to ensure that an observed activity is authentic including running parallel reactions with cells that lack the reporter construct or by not contacting a cell harboring the reporter construct with test compound.

Certain screening methods involve screening for a compound that modulates gene expression normally regulated by CMKLR1 signaling. In certain embodiments, a cell-based assay is conducted in which a cell expressing CMKLR1 is contacted to a candidate agent (e.g., a CMKLR1 binding agent) and monitored for changes in gene expression that are similar, or substantially similar, to those induced by a natural ligand for CMKLR1. In certain other embodiments, a cell-based assay is conducted in which a cell expressing CMKLR1 is contacted to its natural ligand and a candidate agent and monitored for perturbations in gene expression. By perturbations in gene expression is meant that the gene expression changes induced by a CMKLR1 ligand binding to CMKLR1 is altered when the candidate agent is present.

Certain screening methods involve screening for a compound that modulates CMKLR1 signaling events when contacted to a cell expressing CMKLR1. These assays can be carried out in the presence or absence of a natural ligand for CMKLR1. Such methods generally involve monitoring for modulation of downstream signaling events as described above, e.g., protein phosphorylation, GDP/GTP exchange, etc.

Compounds can also be further validated as described below.

Compounds that are initially identified by any of the foregoing screening methods can be further tested to validate their apparent activity. The basic format of such methods involves administering a lead compound identified during an initial screen to an animal that serves as a model for humans. The animal models utilized in validation studies generally are mammals. Specific examples of suitable animals include, but are not limited to, primates, mice, and rats.

Active test agents identified by the screening methods described herein that modulate CMKLR1 activity can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

A functional assay that detects leukocyte chemotaxis may be used for confirmation. For example, a population of cells that demonstrate chemerin chemotaxis (e.g., dendritic cells or monocyte/macrophages) may be stimulated with chemerin and/or the candidate modulating agent. An agent that antagonizes CMKLR1 activity will cause a decrease in the locomotion of the cells in response to chemerin. An agent that potentiates CMKLR1 activity will act as a chemotaxis factor in the absence of chemerin and/or increase the chemotactic response induced by chemerin. Chemotaxis assays that find use in these methods are known in the art, examples of which are described in U.S. patent application Ser. No. 10/958,527, entitled “Family of Cystatin-Related Chemoattractant Proteins” (incorporated herein by reference in its entirety). An agent that is a chemoattractant inhibitor will decrease the concentration of cells at a target site of higher concentration of chemerin.

### Antibodies

In some embodiments, the CMKLR1 modulator is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The term includes monoclonal antibodies, multispecific antibodies (antibodies that include more than one domain specificity), human antibody, humanized antibody, and antibody fragments with the desired biological activity.

The specific or selective fit of a given structure and its specific epitope is sometimes referred to as a “lock and key” fit. The archetypal antibody molecule is the immunoglobulin, and all types of immunoglobulins, IgG, e.g. IgG1, IgG2a, IgG2b, IgG3, IgG4, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be polyclonal antibodies, although monoclonal antibodies are preferred because they may be reproduced by cell culture or recombinantly, and can be modified to reduce their antigenicity. Such antibodies are well known in the art and commercially available, for example from Research Diagnostics, Becton Dickinson, etc.

Polyclonal antibodies can be raised by a standard protocol by injecting a production animal with an antigenic composition, formulated as described above. See, e.g., Harlow and Lane, Antibodies: A Laboratory Manual, Cold Spring Harbor Laboratory, 1988. In one such technique, CMKLR1, its ligand, or an antigenic portion of thereof (e.g., a peptide), is initially injected into any of a wide variety of mammals (e.g., mice, rats, rabbits, sheep or goats). When utilizing an entire protein, or a larger section of the protein, antibodies may be raised by immunizing the production animal with the protein and a suitable adjuvant (e.g., Fruend's, oil-in-water emulsions, etc.) When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions, a superior immune response may be elicited if the polypeptide is joined to a carrier protein, such as ovalbumin, BSA or KLH. The peptide-conjugate is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. The immortal cell line utilized is preferably selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

Preferably, the immortal fusion partners utilized are derived from a line that does not secrete immunoglobulin. The resulting fused cells, or hybridomas, are cultured under conditions that allow for the survival of fused, but not unfused, cells and the resulting colonies screened for the production of the desired monoclonal antibodies. Colonies producing such antibodies are cloned, expanded, and grown so as to produce large quantities of antibody, see Kohler and Milstein, 1975 Nature 256:495 (the disclosures of which are hereby incorporated by reference).

Large quantities of monoclonal antibodies from the secreting hybridomas may then be produced by injecting the clones into the peritoneal cavity of mice and harvesting the ascites fluid therefrom. The mice, preferably primed with pristane, or some other tumor-promoter, and immunosuppressed chemically or by irradiation, may be any of various suitable strains known to those in the art. The ascites fluid is harvested from the mice and the monoclonal antibody purified therefrom, for example, by CM Sepharose column or other chromatographic means. Alternatively, the hybridomas may be cultured in vitro or as suspension cultures. Batch, continuous culture, or other suitable culture processes may be utilized. Monoclonal antibodies are then recovered from the culture medium or supernatant.

In addition, the antibodies or antigen binding fragments may be produced by genetic engineering. In this technique, as with the standard hybridoma procedure, antibody-producing cells are sensitized to the desired antigen or immunogen. The messenger RNA isolated from the immune spleen cells or hybridomas is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones, which co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell). When antibody gene synthesis is induced in the transfected host, the heavy and light chain proteins self-assemble to produce active antibodies that can be detected by screening with the antigen or immunogen.

Antibodies with a reduced propensity to induce a violent or detrimental immune response in humans (such as anaphylactic shock), and which also exhibit a reduced propensity for priming an immune response which would prevent repeated dosage with the antibody are preferred for use in the invention. Thus, humanized, single chain, chimeric, or human antibodies, which produce less of an immune response when administered to humans, are preferred for use in the present invention. Also included in the invention are multi-domain antibodies.

A chimeric antibody is a molecule in which different portions are derived from different animal species, for example those having a variable region derived from a murine mAb and a human immunoglobulin constant region. Techniques for the development of chimeric antibodies are described in the literature. See, for example, Morrison et al. (1984) Proc. Natl. Acad. Sci. 81:6851-6855; Neuberger et al. (1984) Nature 312:604-608; Takeda et al. (1985) Nature 314:452-454. Single chain antibodies are formed by linking the heavy and light chain fragments of the Fv region via an amino acid bridge, resulting in a single chain polypeptide. See, for example, Huston et al., Science 242:423-426; Proc. Natl. Acad. Sci. 85:5879-5883; and Ward et al. Nature 341:544-546.

Antibody fragments that recognize specific epitopes may be generated by techniques well known in the field. These fragments include, without limitation, F(ab′)₂ fragments, which can be produced by pepsin digestion of the antibody molecule, and Fab fragments, which can be generated by reducing the disulfide bridges of the F(ab′)₂ fragments.

In one embodiment of the invention, a human or humanized antibody is provided, which specifically binds to the extracellular region of CMKLR 1 with high affinity. In another embodiment, a human or humanized antibody is provided, which specifically binds to a CMKLR1 ligand (e.g., chemerin).

Alternatively, polyclonal or monoclonal antibodies may be produced from animals that have been genetically altered to produce human immunoglobulins. Techniques for generating such animals, and deriving antibodies therefrom, are described in U.S. Pat. Nos. 6,162,963 and 6,150,584, incorporated fully herein by reference.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ficin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

Candidate CMKLR1, or ligand, antibodies can be tested for by any suitable standard means, e.g. ELISA assays, etc. As a first screen, the antibodies may be tested for binding against the immunogen, or against the entire polypeptide. As a second screen, anti-CMKLR1 candidates may be tested for binding to a tissue expressing CMKLR1. For these screens, the anti-CMKLR1 candidate antibody may be labeled for detection. After selective binding is established, the candidate antibody, or an antibody conjugate may be tested for appropriate activity (e.g., the ability to regulate fat accumulation) in an in vivo model. Other properties of the candidate CMKLR1 modulating agent can be determined. These include, but are not limited to, measuring binding affinity to a target, biodistribution of the compound within an animal or cell, etc. These and other screening methods known in the art provide information on the ability of a compound to bind to, modulate, or otherwise interact with the specified target and are a measure of the compound's efficacy.

Representative CMKLR1, or ligand inhibitory agents also include, but are not limited to: antisense oligonucleotides, and the like. The antisense reagent may be antisense oligonucleotides (ODN), particularly synthetic ODN having chemical modifications from native nucleic acids, or nucleic acid constructs that express such antisense molecules as RNA. The antisense sequence is complementary to the targeted CMKLR1, or ligand, and inhibits its expression. One or a combination of antisense molecules may be administered, where a combination may comprise multiple different sequences.

Antisense molecules may be produced by expression of all or a part of the target CMKLR1, or ligand sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 25, usually not more than about 23-22 nucleotides in length, where the length is governed by efficiency of inhibition, specificity, including absence of cross-reactivity, and the like.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature that alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′—NH-5′-O-phosphoroamidate. Peptide nucleic acids replace the entire ribose phosphodiester backbone with a peptide linkage. Sugar modifications are also used to enhance stability and affinity. The alpha-anomer of deoxyribose may be used, where the base is inverted with respect to the natural beta-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Anti-sense molecules of interest include antagomir RNAs, e.g. as described by Krutzfeldt et al., supra., herein specifically incorporated by reference. Small interfering double-stranded RNAs (siRNAs) engineered with certain ‘drug-like’ properties such as chemical modifications for stability and cholesterol conjugation for delivery have been shown to achieve therapeutic silencing of an endogenous gene in vivo. To develop a pharmacological approach for silencing miRNAs in vivo, chemically modified, cholesterol-conjugated single-stranded RNA analogues complementary to miRNAs were developed, termed ‘antagomirs’. Antagomir RNAs may be synthesized using standard solid phase oligonucleotide synthesis protocols. The RNAs are conjugated to cholesterol, and may further have a phosphorothioate backbone at one or more positions.

Also of interest in certain embodiments are RNAi agents. In representative embodiments, the RNAi agent targets the CMKLR1, or ligand genetic sequence. By RNAi agent is meant an agent that modulates expression of CMKLR1, or ligand by a RNA interference mechanism. The RNAi agents employed in one embodiment of the subject invention are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

In certain embodiments, instead of the RNAi agent being an interfering ribonucleic acid, e.g., an siRNA or shRNA as described above, the RNAi agent may encode an interfering ribonucleic acid, e.g., an shRNA, as described above. In other words, the RNAi agent may be a transcriptional template of the interfering ribonucleic acid. In these embodiments, the transcriptional template is typically a DNA that encodes the interfering ribonucleic acid. The DNA may be present in a vector, where a variety of different vectors are known in the art, e.g., a plasmid vector, a viral vector, etc.

### EXPERIMENTAL

### Chemerin: a Novel Adipokine that Regulates Adipogenesis and Adipocyte Metabolism

Chemerin (RARRES2 or TIG2) is a recently discovered chemoattractant protein that serves as a ligand for the G protein-coupled receptor CMKLR1 (ChemR23 or DEZ) and has a role in adaptive and innate immunity. Here we show an unexpected, high-level expression of chemerin and its cognate receptor CMKLR1 in mouse and human adipocytes. Cultured 3T3-L1 adipocytes secrete chemerin protein, which triggers CMKLR1 signaling in adipocytes and other cell types and stimulates chemotaxis of CMKLR1-expressing cells. Adenoviral shRNA targeted knockdown of chemerin or CMKLR1 expression impairs differentiation of 3T3-L1 cells into adipocytes, reduces the expression of adipocyte genes involved in glucose and lipid homeostasis and alters metabolic functions in mature adipocytes. We conclude that chemerin is a novel adipose derived signaling molecule that regulates adipogenesis and adipocyte metabolism.

Initial screening studies in our laboratory identified high expression of chemerin and CMKLR1 in white adipose tissue of mouse. These data suggested that adipose tissue is a source and target for chemerin signaling. We have proposed and tested the hypothesis that chemerin is an adipokine with a regulatory role in adipogenesis or adipocyte function.

Animal protocol and housing. The Dalhousie University Committee on Laboratory Animals approved experimental procedures involving mice according to the guidelines of the Canadian Council on Animal Care. C57/B6/J mice were bred in house, in the Carleton Campus Animal Care Facility. The mice were kept on a 12-hour day/night cycle, were housed in cages lined with pine bedding and had free access to water and Purina mouse chow.

RNA isolation and QPCR analysis. Mice were anaesthetized with 50 mg kg⁻¹ of sodium pentobarbital. Tissues were isolated and snap frozen in liquid nitrogen. For adipose tissue fractionation, freshly isolated epididymal fat pads were placed in 5 ml of ice-cold DMEM with 1% BSA and 2 mg/ml of collagenase II and minced with scissors. The tissue was incubated for 15 min at 37° C. with intermittent pipetting, diluted into 10 ml of ice-cold DMEM and filtered through 70 μM nylon mesh and centrifuged at 2200 RPM for 5 min to separate the adipocyte (buoyant) and stromal vascular fractions (pellet). Total RNA was isolated from each fraction using the Rneasy mini kit (Qiagen, Mississauga, ON) according to the manufacturer's instruction. Total RNA from tissues (5 μg) or cells (0.5 or 1.0 μg) was reverse transcribed using Stratascript™ Reverse Transcriptase (Stratagene, Cedar Creek, Tex.) and 1 μl of the cDNA product was amplified by quantitative-PCR using 125 nM gene specific primers (Table 1) in a total volume of 20 μl with Brilliant SYBR Green QPCR Master Mix (Stratagene) using a Stratagene MX3000p thermocycler. Relative gene expression was normalized to ribosome polymerase II (rpII) or Cyclophilin A (CycA) expression using the ΔΔC_{T} method.

TABLE 1

Quantitative PCR (QPCR) primers

| | | Pro- |
|---|---|---|
| Gene Accession | PCR Primers 5′ to 3′ | duct |
| # | direction | size |
| madiponectin | Fw agccgcttatatgtatcgctca | 118 bp |
| NM_009605.3 | Rv tgccgtcataatgattctgttgg | |
| mchemerin | Fw tacaggtggctctggaggagttc | 195 bp |
| NM_027852.1 | Rv cttctcccgtttggtttgattg | |
| mCMKLR1 | Fw caagcaaacagccactacca | 224 bp |
| NM_008153 | Rv tagatgccggagtcgttgtaa | |
| mcyclophilinA | Fw gagctgtttgcagacaaagttc | 124 bp |
| X52803.1 | Rv ccctggcacatgaatcctgg | |
| mDGAT2 | Fw tctctgtcacctggctcaac | 137 bp |
| NM_026384.2 | Rv gcagtctgtgcagaaggtgt | |
| mFAS | Fw ggaggtggtgatagccggtat | 139 bp |
| NM_007988.2 | Rv tgggtaatccatagagcccag | |
| mGPAT | Fw ctctgtcgtccaacaccatc | 116 bp |
| NM_008149.2 | Rv ctcgttcttcttgggctttc | |
| mGLUT4 | Fw actcttgccacacaggctct | 173 bp |
| NM_009204.1 | Rv aatggagactgatgcgctct | |
| mHSL | Fw gcttggttcaactggagagc | 279 bp |
| NM_010719.5 | Rv gcctagtgccttctggtctg | |
| mlL-6 | Fw tagtccttcctaccccaatttcc | 75 bp |
| NM_031168.1 | Rv ttggtccttagccactccttc | |
| mlNSR | Fw cctgtaccctggagaggtgt | 246 bp |
| NM_010568.1 | Rv cggatgactgtgagatttgg | |
| mleptin | Fw gagacccctgtgtcggttc | 138 bp |
| NM_008493.3 | Rv ctgcgtgtgtgaaatgtcattg | |
| mmac-1β-sub- | FW gtggtgcagctcatcaagaa | 196 bp |
| unit M31039.1 | RV gccatgacctttacctggaa | |
| mperilipin | Fw acactctccggaacaccatc | 116 bp |
| NM_175640.1 | Rv ccctccctttggtagaggag | |
| mPPARγ | Fw tcgctgatgcactgcctatg | 102 bp |
| NM_011146.1 | Rv gagaggtccacagagctgaat | |
| mrpII U37500.1 | Fw ctggacctaccggcatgttc | 132 bp |
| | Rv gtcatcccgctcccaacac | |
| mTNFα | Fw ccctcacactcagatcatcttct | 60 bp |
| NM_013693.1 | Rv gctacgacgtgggctacag | |
| hChemerin | Fw tggaagaaacccgagtgcaaa | 127 bp |
| NM_002889.2 | Rv agaacttgggtctctatgggg | |
| hCMKLR1 | Fw atggactaccactgggttttcggg | 231 bp |
| NM_004072 | Rv gaagacgagagatggggaactcaag | |
| hCyclophilinA | FW ttcatctgcactgccaagac | 158 bp |
| NM_021130.2 | RV tcgagttgtccacagtcagc | |
| hLeptin | FW ggctttggccctatcttttc | 198 bp |
| NM_000230 | RV ccaaaccggtgactttctgt | |
| hPPARγ | FW gagcccaagtttgagtttgc | 198 bp |
| NM_138712 | RV ctgtgaggactcagggtggt | |

Abbreviations, Mouse (m) and human (h). DGAT2, diacylglycerol o-acetyltransferase 2; FAS, fatty acid synthase; GLUT4, facilitated glucose transporter member 4; GPAT, glycerol-3-phosphate acyltransferase; HSL, hormone sensitive lipase; IL-6, interleukin-6; INSR, insulin receptor; PPARγ, peroxisome proliferator activated receptor; RPII; ribosome polymerase II; TNFα, tumor necrosis factorα.

TABLE 1b

shRNAi oligonuleotides
Gene Accession # Primers 5′ to 3′ direction

mchemerin NM_027852
FW:
ACCGGATAGTCCACTGCCCAATTCCGAAGAATTGGGCAGTGGACTATCC
Rw:
AAAAGGATAGTCCACTGCCCAATTCTTCGGAATTGGGCAGTGGACTATCC
mCMKLR1 NM_008153
Fw:
CACCGGAAGATAACCTGCTTCAACACGAATGTTGAAGCAGGTTATCTTCC
Fw:
AAAAGGAAGATAACCTGCTTCAACATTCGTGTTGAAGCAGGTTATCTTCC

ShRNA primers were synthesized in the sense-loop-antisense orientation. The bold and underlined regions correspond to the target chemerin and CMKLR1 gene sequences.

Mouse adipocyte cell culture. 3T3-L1 preadipocytes were obtained from the American Tissue Culture Collection (Manassas, Va.) and grown according to standard protocols. Confluent preadipocytes were differentiated in adipocyte media (DMEM, 10% FBS, 850 nM insulin and 0.1% penicillin/streptomycin) supplemented with 250 nM dexamethasone and 100 μM isobutylmethylxanthine (IBMX) for 3 days. After this time, the cells were maintained in adipocyte media, which was changed every 2 days. All media was phenol red free. Adipocyte conditioned media used for western blots, CMKLR1 activation or the chemotaxis assay was obtained by replacing the regular adipocyte media with serum free adipocyte media for a period of 24 hr. For ERK1/ERK2 phosphorylation studies, adipocyte media was replaced with fresh media 4 hour prior to the assay. At the time of the assay fresh adipocyte media containing (0.2, 1.0 or 10 nM) chemerin was added to the cells. Between 2 and 30 min thereafter, media was removed and 100 μl of 1× Laemmli buffer was added to stop the reaction and lyse the adipocytes. Oil Red O staining of adipocytes and quantification of extracted dye was carried out as previously described.

Western blotting. We generated polyclonal rabbit antibodies against a synthetic peptide region of mouse chemerin. The immunizing peptide (CLAFQEIGVDRAEEV) corresponded to amino acids 47-60 of the predicted mouse chemerin protein and was conjugated through a non-native N-terminal cysteine to keyhole limpet hemocyanin (KLH) (Sigma Genosys Canada, Oakville, ON). Rabbits were given a primary immunization by subcutaneous injection of 150 μg of the peptide-KLH conjugate in Freund's complete adjuvant. Subsequent immunizations with the peptide-KLH conjugate in Freund's incomplete adjuvant were administered 3, 5 and 7 weeks later. 10 days after the final immunization the rabbits were exsanguinated by cardiac puncture. The specificity of the antiserum was confirmed by testing immunoreactivity against recombinant mouse chemerin (R&D Systems, Minneapolis, Minn.) and COS7 cells that were transiently transfected with a mchemerin-pFlag-CMV-5a construct or the control vector pFlag-CMV-5a (Stratagene). For western blots, 100 μl of 24 hr conditioned adipocyte media was added to 20 μL of 6×SDS loading buffer. Fifteen μl of the solution was separated on a 12.5% polyacrylamide gel and transferred overnight to a nitrocellulose membrane. Blots were blocked (1 hr) in 3% skim milk in pH 7.5 tris buffered saline with 0.05% tween (TBST), incubated with protein A purified rabbit anti-chemerin:antiserum (1:200) in 3% skim-milk-TBST for 2 hr at RT and then horseradish peroxidase conjugated mouse anti-rabbit IgG secondary antibody (1:25,000) for 1 hr at RT in 3% skim-milk-TBST. Immunoreactivity was detected by incubation with Fluorescent ECL-plus™ reagent (GE Healthcare, Piscataway, N.J.) and visualized directly with a Storm 840 phosphor imager (GE Healthcare). A similar protocol using 1:200 dilutions of P-ERK (E-4) and ERK (D-2) antibodies (Santa Cruz Biotechnology, Santa Cruz, Calif.) was used to detect endothelial related kinase 2 (ERK2) and phosphorylated ERK1 and ERK 2 in whole cell lysates from chemerin treated adipocytes.

Aequorin assay. The aequorin assay is a cell-based, bioluminescence reporter gene assay used to detect CMKLR1 activation. CMKLR1-CHO-K1 cells (Euroscreen, Belgium) express human CMKLR1, G₍₁₆ (G protein) and an intracellular reporter gene (mitochondrial aequorin) that is activated by the Ca²⁺ influx produced when chemerin binds to CMKLR1 activating G_{α16}. The control CHO-K1 cells (Euroscreen, Belgium) express the G_{α16} protein and mitochondrial aequorin only and do not respond to chemerin. Control CHO-K1 cells were maintained in complete Ham's F12 with 10% FBS, 100 IU/ml penicillin, 100 μg/ml streptomycin, 250 μg/ml Zeocin. This media supplemented with the antibiotic G418 (400 μg/ml) was used for maintenance of the CHOCMKLR1 cells. Cells in mid-log phase, grown in media without antibiotics for 20 hr prior to the test were detached with PBS containing 5 mM EDTA, centrifuged and suspended (5×10⁶ cells/ml) in 0.1% BSA media (DMEM/HAM's F12 with HEPES, without phenol red) plus 5 μM Coelenterazineh (Sigma Aldrich, Oakville, ON) and incubated for 4 hr at room temperature (RT) on an orbital shaker. Cells were then diluted 1 in 10 with 0.1% BSA media and incubated for 1 hr. For each measurement 50 μl of cell suspension (25,000 cells) was added into each well of the plate containing 50 μl of diluted conditioned media or recombinant chemerin. The emitted light was recorded for 20 s at 469 nM following the injection of cells. The intensity of emitted light was normalized to the response produced by the Ca₂₊ ionophore digitonin (50 μM).

Cell migration assay. Conditioned serum free media from preadipocytes and adipocytes was tested for the ability to stimulate migration of the murine pre-B lymphoma cell line L1.2 stably transfected with human CMKLR1 (L1.2-CMKLR1) or empty vector (L1.2-pcDNA3). The cells were maintained as previously described. All assay incubations were performed under standard conditions (37° C. in 95% air, 5% CO₂). The L1.2 cells were plated at a density of 1×10⁶ cells/ml and were treated with 5 mM n-butyric acid 24 hr prior to experimentation. Purified mouse chemerin and conditioned (24 hr) serum free media samples from preadipocyte or 13-day mature adipocyte cell cultures was diluted 1:100 into the chemotaxis medium (600 μl final volume) consisting of phenol red-free RPMI 1640 and 1% fetal bovine serum and was incubated for 30 min under standard conditions. Transwell inserts (5 μm pore size) containing 250,000 L1.2-CMKLR1 or L1.2-pcDNA cells in 100 μl of chemotaxis media were added to wells containing the media samples and incubated for 3 hr. The inserts were removed and cells that migrated into the lower chamber were labeled for 3 hr with calcein-AM. The calcein-AM labeled cells were diluted 1:10 in PBS and fluorometrically measured (485 nm excitation and 520 nm emission) using a Carey spectrofluorometer. A standard curve was generated from calcein-AM labeling of known quantities of either cell type and was used to quantify the total number of cells (% input migration) that migrated towards the test samples.

Immunohistochemistry. Preadipocytes were plated on collagen-coated glass coverslips and differentiated according to the standard protocol. On day 8, the cells were rinsed in PBS and fixed in 4% paraformaldehyde. Fixed cells were rinsed in PBS and incubated with 0.1% triton-x 100 (3 min) followed by incubation in standard blocking solution (10% goat serum, 1% bovine serum albumin in phosphate buffered saline) for 1 hr, 1:200 dilution of anti-human CMKLR1 monoclonal antibody (clone 84939, R&D Systems) for 2 hr, 1/200 dilution of Alexa Fluor 488 goat anti-mouse IgG₃ (Invitrogen, Burlington, ON) for 1 hr and counterstained with Hoechst 33258 (Sigma) 1 μg/ml in PBS for 5 min. Slides were washed in PBS and mounted with DakoCytomation fluorescent mounting medium (DakoCytomation Carpinteria, Calif.). Images were captured on a Zeiss Axiovert 200 Inverted Microscope equipped with an AxioCam camera system (Zeiss Canada, Toronto, ON).

Adenoviral small hairpin loop RNA interference (ShRNA). ShRNA vectors were constructed using the Block-it™ Adenoviral RNAi expression system (Invitrogen) according to the manufacturers' instructions. Single stranded oligonucleotides for shRNA were designed using Block-it™ RNAi designer (Invitrogen). Positive adenoviral clones, mchemerin-pAD-shRNA (CE-shRNA), mCMKLR1-pAD-shRNA (CR-shRNA) and LacZ-pAD-shRNA (LZ-shRNA) were amplified in the HEK-293A producer cell line. Viral copy number in crude lysates was determined by quantitative PCR amplification. A poly-l-lysine (MW 30,000-70,000) assisted procedure was used to transduce confluent preadipocytes or adipocytes. Crude adenoviral lysates (MOI 100-1000) were added to poly-l-lysine (0.5 μg/ml) optimem mix and incubated for 100 min at RT. The MOI refers to the ratio of viral copy/cell number. One-day post-confluent preadipocytes were washed once with PBS and 500 [symbol] l of the transduction mix was added to each well (12-well plate) and incubated under standard conditions for 2 hr followed by addition of 1 ml of DMEM with 0.2% BSA and incubated overnight. The next day media was replaced with normal preadipocyte media for 6 hr. At this time the normal adipocyte differentiation and maintenance protocol was followed. Using the same protocol adipocytes at day 4 post-differentiation were transduced with the crude adenoviral lysates. For the lipolysis assays, day 7 adipocytes were switched to DMEM+0.1% BSA with or without 0.2 or 1.0 nM chemerin. 24 hr later, media was replaced with DMEM+0.1% BSA with or without 2 μM isoproterenol or 100 μM IBMX. Glycerol released into the media over a period of 2 or 4 hr was measured using a lipoysis assay kit (ZenBio) according to the product instructions.

Adiponectin measurements. An enzyme-linked immunosorbant assay (adiponectin, Quantikine kit, R&D systems, Minneapolis, Minn.) was used to measure adiponectin levels in 24-hr conditioned serum-free media from adipocytes 5 or 8 days post-differentiation.

Human cells and RNA. Human preadipocytes and adipose tissue RNA were commercially available (ZenBio, Chapel Hill, N.C.). Human liver and placenta RNA were purchased from Stratagene. Superlots of cryopreserved human subcutaneous preadipocytes (ZenBio Inc.) contained preadipocytes pooled from 6 female donors aged 35-45 with an average BMI of 29. The preadipocytes were seeded on 12-well plates according to the manufacturers' instructions. To induce differentiation, the preadipocytes were incubated with 1 ml of adipocyte differentiation media (ZenBio, Research Triangle Park, NC) for 7 days. Thereafter, adipocytes were maintained in DMEM/F12 with 10% FBS, 850 nM insulin. Oil Red O staining and preparation of cells for RNA extraction was performed as described for the 3T3-L1 cells.

Statistical analysis. All data are expressed as mean ±s.e.m. of 3-4 separate measurements unless otherwise stated in the figure legends. A one or two-way analysis of variance (ANOVA) was used for multiple comparison procedures. A Tukey's test was used for post-hoc analysis of the significant ANOVA. A difference in mean values between groups was considered to be significant when p δ 0.05.

Results

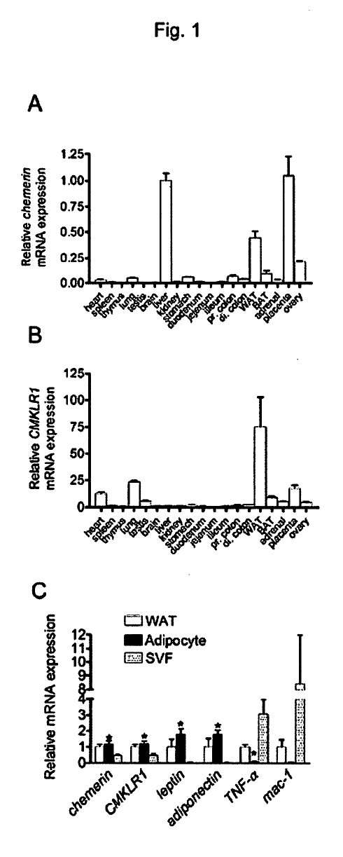

Using quantitative real-time PCR analysis, it was determined that murine chemerin mRNA was most highly expressed in white adipose tissue, liver and placenta with intermediate expression in ovary and brown adipose tissue (FIG. 1A). Chemerin mRNA levels in other tissues were less than 5% of that in liver. Expression of CMKLR1 mRNA was highest in white adipose tissue, followed by intermediate levels in lung, heart and placenta (FIG. 1B). By comparison, CMKLR1 expression in other tissues was very low. Within white adipose tissue, chemerin and CMKLR1 expression was enriched 2-fold in adipocytes compared to the stromal vascular fraction (FIG. 1C). Differential expression of the adipocyte markers leptin and adiponectin and stromal vascular expressed genes tnf-α and mac-1 confirmed effective separation of adipocytes from stromal vascular cells.

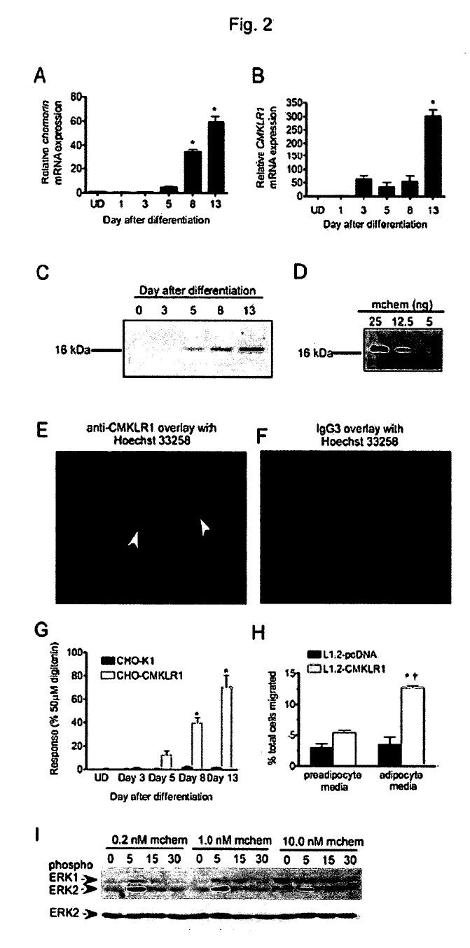

Based on these gene expression data and previous observations that chemerin is a secreted protein, we believe that adipocytes are a source and target for chemerin signaling. This was tested using the well-established 3T3-L1 adipocyte cell culture mode. Chemerin expression was lowest in undifferentiated cells but increased dramatically with adipocyte differentiation and by day-13 was 60-fold higher as compared to undifferentiated cells (FIG. 2A). Similarly, CMKLR1 expression was lowest in undifferentiated 3T3-L1 cells but increased progressively to levels 300-fold higher in 13-day differentiated cells versus undifferentiated cells (FIG. 2B). Overall, chemerin and CMKLR1 exhibited a similar temporal pattern of expression to that of the established adipocyte differentiation markers PPARγ and leptin.

Chemerin is believed to be secreted as an 18 kDa inactive pro-protein that undergoes extracellular serine protease cleavage of the C-terminal portion of the peptide to generate the 16 kDa active chemerin. A protein corresponding to this mature form of chemerin was detected by Western blotting of conditioned 3T3-L1 adipocyte media as early as day 5 after differentiation. Consistent with the mRNA levels, chemerin secretion increased with adipogenesis (FIG. 2C,D). Histological analysis of mature adipocytes (day 8) with an anti-CMKLR1 antibody demonstrated intense immunoreactivity (FIG. 2E) localized to the cell periphery (white arrow heads). Counterstaining of cell nuclei with Hoescht 33258 confirmed that anti-CMKLR1 immunoreactivity was localized to the non-nuclear regions of these cells. Immunofluorescence was virtually undetectable in cells incubated with the IgG₃ control antibody (FIG. 2F). The differentiation-dependent expression and secretion of proteolytically processed chemerin thus strongly support an adipokine-like function for this protein.

To address the biological activity, we used an aequorin cell-based, reporter-gene assay to measure activation of CMKLR1 (Wittamer et al. (2003) JEM 198:977-985). Both human (Kₘ=48±12 pM) and mouse (Kₘ=119±29 pM) chemerin were potent activators of the CMKLR1-aequorin reporter assay). Using this assay, activation of CMKLR1 by 3T3-L1 adipocyte-conditioned media was detected as early as 3 days after inducing differentiation and increased further with adipocyte maturation (FIG. 2G). This result was consistent with the differentiation-dependent expression of chemerin mRNA and secreted protein.

Chemerin stimulates chemotaxis of dendritic cells and macrophages that express CMKLR1 and may be responsible for recruitment of these cells to sites of inflammation. Using a chemotaxis chamber assay, we determined that media (1:100 dilution) from 13-day adipocytes produces a 4-fold higher migration of CMKLR1-expressing L1.2 cells as compared to pcDNA-transfected L1.2 cells (FIG. 2H). Migration of CMKLR1-expressing L1.2 cells towards adipocyte media was 3-fold higher than the basal migration towards preadipocyte media. Consistent with these data, recombinant mouse chemerin (0.1 to 1 nM) stimulated migration of CMKLR1 expressing L1.2 cells in a dose-dependent fashion but had no effect on empty vector pcDNA-transfected L1.2 cells. These observations confirmed the presence of functionally active chemerin in adipocyte cell culture media.

A number of adipokines act in a local autocrine/paracrine fashion to regulate adipogenesis and adipocyte metabolism. While chemerin signaling pathways are not well established, CMKLR1 activation is reported to increase intracellular Ca²⁺ concentrations and phosphorylation of p42 (ERK2) and p44 (ERK1) mitogen activated protein kinases (MAPKs). This latter effect may be relevant to adipocyte function as ERK1/2 signaling is involved in adipogenesis and lipolysis pathways. Thus, we used ERK1/2 phosphorylation to determine if adipocytes were responsive to chemerin. Mouse chemerin (0.2 nM) applied to adipocytes transiently and reversibly stimulated (4-5 fold) ERK1/2 phosphorylation (FIG. 2I). At higher (1 or 10 μM) concentrations, chemerin produced a lower response and suggests desensitization and/or inhibition of signaling at higher concentrations. Non-phosphorylated ERK2 served as a loading control and its expression was similar in all groups. These findings strongly support a potential local/autocrine signaling effect of adipocyte-derived chemerin.

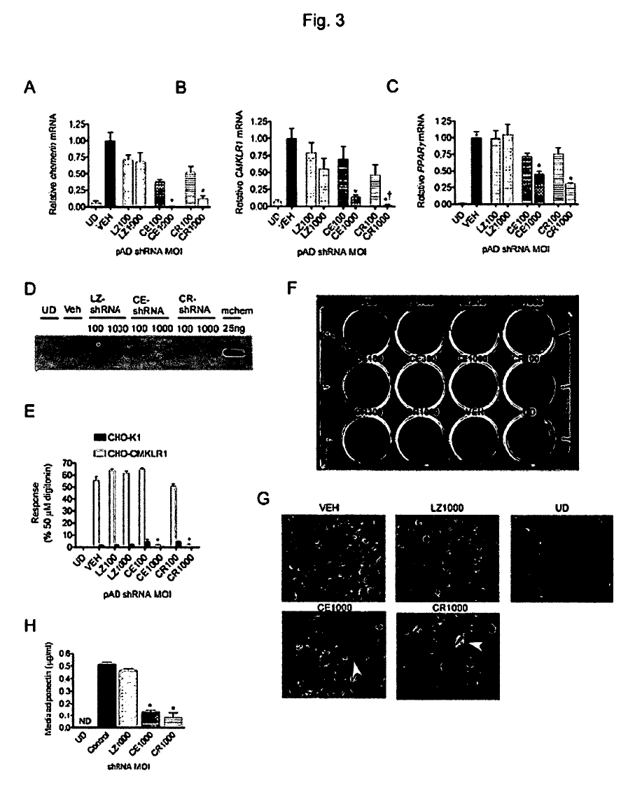

Given the profound increase of chemerin secretion and CMKLR1 expression early in adipocyte differentiation, we hypothesized that an autocrine/local function of this signaling pathway is to regulate adipocyte differentiation. To address this, confluent preadipocytes were transduced with adenoviral vectors expressing shRNA for chemerin (CE), CMKLR1 (CR) or LacZ (LZ; to control for non-specific effects of viral transduction and shRNA expression) for a period of 24 hr. After this time, differentiation media was added to the cells and differentiation was allowed to proceed as normal. Consistent with our earlier experiments, chemerin and CMKLR1 expression were about 15-fold higher in the day 5 nontransduced vehicle (VEH) control cells compared to the undifferentiated cells. CE- or CR-shRNA transduction of preadipocytes produced a dose-dependent reduction of the mRNA level of the respective target genes (FIG. 3A,B) as well as secretion of bioactive chemerin into media (FIG. 3D,E) compared to VEH-treated or LZ-transduced cells. Expression of PPARγ (FIG. 3C) as well as Oil Red O staining of neutral lipid (FIG. 3F) measured 8 days after inducing differentiation was also markedly reduced by 1000 MOI CE and CR-shRNA treatments.

Phase contrast images of live unstained cells taken at day 3, 4, 5 and 8 after inducing differentiation demonstrate the overall time course of cellular changes during the differentiation period. Morphological changes produced by CMKLR1 and chemerin shRNA treatment were obvious by day 4 after inducing differentiation. By day 8, it was readily apparent that cells treated with CE-shRNA remained primarily fibroblast-like (white arrow; FIG. 3G) whereas cells treated with CR-shRNA displayed a mixture of abnormally large cells with perinuclear lipid accumulation (black arrow) and fibroblast-like cells (white arrow). Adiponectin secretion into adipocyte media was also reduced by CE- and CR-shRNA treatment (FIG. 3H). By comparison, LZ-shRNA treatment did not affect any of these parameters, indicating that the adenoviral transduction alone did not affect the adipocyte differentiation program.

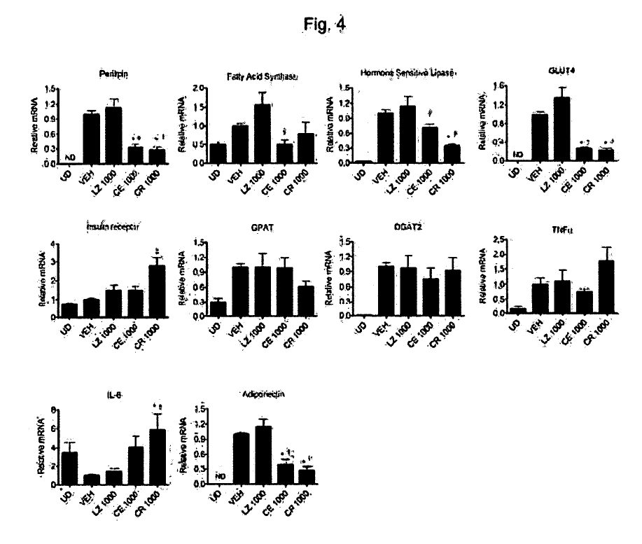

Consistent with the abrogation of adipocyte differentiation, the expression of a number of genes was reduced by these treatments (FIG. 4). Chemerin and CMKLR1 knockdown decreased perilipin (60%), glucose transporter-4, (GLUT4; 80%), adiponectin (50-75%) and hormone sensitive lipase (HSL; 40-60%) expression compared to vehicle and/or LZ control vector. Interestingly, CMKLR1 knockdown increased the expression of insulin receptor (INSR) and IL-6 compared to vehicle and/or LZ control. Glycerolphosphate acteyltransferase (GPAT), diacylglycerol3-phosphate acteyltransferase-2 (DGAT2) and TNFα expression were unaffected by CMKLR1 or chemerin knockdown, although TNFα displayed a trend towards increased levels in the CR-shRNA treated cells. Fatty acid synthase (FAS) was significantly reduced by CEshRNA treatment as compared to the LZ-shRNA control.

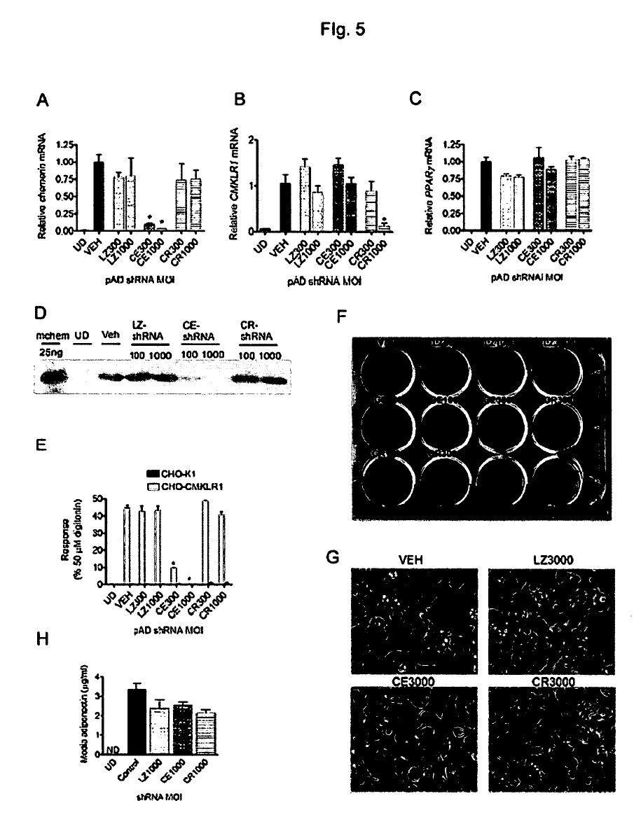

The finding that CE-shRNA treatment partially decreased CMKLR1 expression and that CR-shRNA treatment produced a partial loss of chemerin expression and activity and is also consistent with the impairment of adipocyte differentiation caused by these treatments. Overall, these findings indicated that chemerin/CMKLR1 signaling is critical very early in the adipocyte differentiation process. To further investigate this early requirement for chemerin/CMKLR1 signaling, preadipocytes were incubated for 3 days in differentiation media followed by transduction with the adenoviral shRNA on day 4. When this post-differentiation protocol was followed, CE-shRNA or CR-shRNA reduced chemerin (≧97%) and CMKLR1 (≧85%) mRNA levels respectively, compared to the nontransduced VEH or LZ-shRNA control (FIG. 5A,B). Furthermore, chemerin knockdown at this stage did not alter CMKLR1 expression and CMKLR1 knockdown did not alter chemerin expression. Western blot (FIG. 5D) and aequorin assay (FIG. 5E) for bioactive chemerin in adipocyte media on day-8 post-differentiation confirmed a complete loss of both chemerin protein and activity with CE-shRNA but not with CR-shRNA treatment. In contrast to predifferentiation knockdown, post-differentiation reduction of chemerin or CMKLR1 expression had no overt effect on adipocyte differentiation or phenotype as indicated by PPARγ expression (FIG. 5C), neutral lipid accumulation (FIG. 5F), cell morphology (black arrows; FIG. 5G) or adiponectin secretion (FIG. 5H).

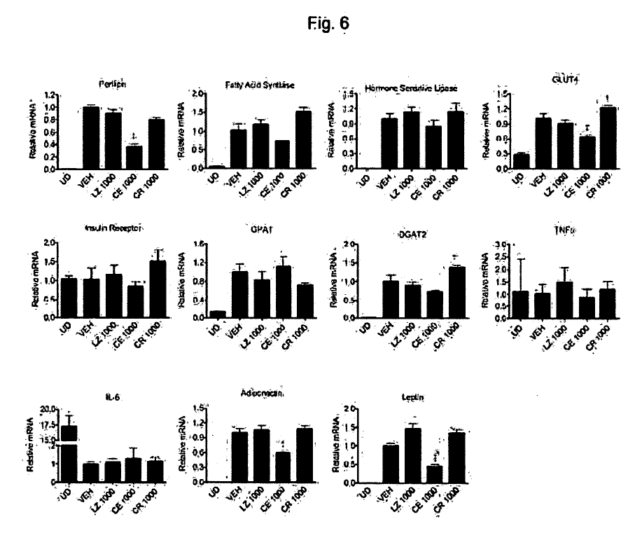

Thus, for normal adipogenesis, there is an essential requirement for chemerin and CMKLR1 within, but not after the first 3-days of the adipocyte differentiation process. While post-differentiation knockdown of chemerin and CMKLR1 had no overt effect on adipocyte phenotype, a number of adipocyte-expressed genes were differentially affected by these treatments (FIG. 6). Chemerin-knockdown reduced perilipin, GLUT4, adiponectin and leptin expression compared to VEH, LZ-shRNA and CR-shRNA treated cells. GLUT4 and DGAT2 expression were higher with CMKLR1 knockdown as compared to the LZ controls. In comparison, HSL, GPAT, IL6 and TNF (were not affected by chemerin or CMKLR1 knockdown. Given the effects on a number of key adipocyte genes, the chemerin-CMKLR1 pathway may modulate the metabolic function of mature adipocytes.

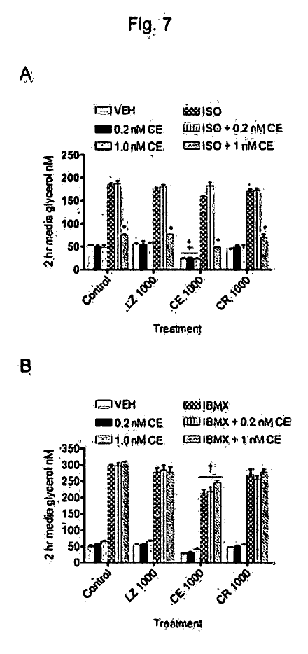

Post-differentiation knockdown of chemerin, but not CMKLR1, reduced basal lipolysis by 50-55% as measured by glycerol release into the adipocyte cell media (FIGS. 7A and 7B). Pre-treatment with 0.2 or 1 nM chemerin for 24 hr did not restore basal lipolysis in the chemerin knockdown cells nor did it alter basal lipolysis in the control, LZshRNA and CR-shRNA treated cells (FIGS. 7A and 7B). Treatment with 2 μM isproterenol stimulated glycerol release into the media to a similar level in each of the treatment groups. Pre-incubation of adipocytes with 1.0 nM chemerin for 24 hr almost completely blocked isoproterenol-stimulated lipolysis in the control, LZ-CE and CR-shRNA-treated cells (FIG. 7A). The phosphodiesterase inhibitor IBMX also stimulated lipolysis. However, IBMX stimulated lipolysis was 25% lower in cells treated with chemerin shRNA compared to VEH and LZshRNA treatment groups. Unlike with isoproterenol treatment, IBMX stimulated-lipolysis was not reduced by chemerin pretreatment (FIG. 7B).

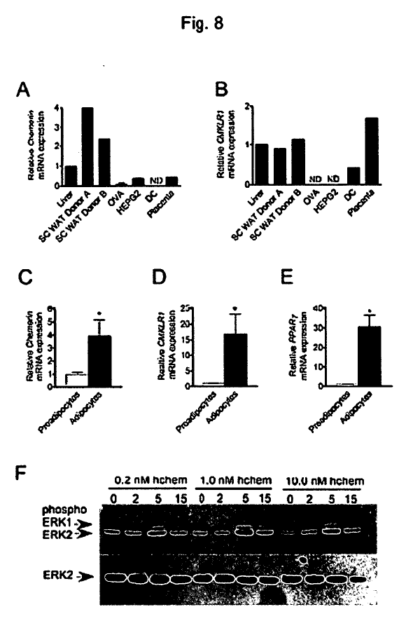

To determine if white adipose expression and function of chemerin and CMKLR1 is conserved and relevant to humans we performed gene expression profiling in human adipose tissues, preadipocytes and adipocytes. Similar to our findings in mouse, chemerin and CMKLR1 were highly expressed in subcutaneous adipose tissue from two human donors (FIG. 8A,B). Comparatively lower expression of chemerin was detected in human liver, ovarian carcinoma cells, hepatic carcinoma cells and placenta but was not detectable in dendritic cells. Subcutaneous white adipose tissue, liver and placenta had similar expression of CMKLR1 mRNA and were higher than expression in dendritic cells. In primary human adipocytes chemerin and CMKLR1 expression was increased 3-fold and 15-fold respectively, as compared to preadipocytes (FIG. 8C,D). Expression of the adipogenesis marker PPARγ was markedly increased (30-fold) in differentiated cells compared to preadipocytes (FIG. 8E). The differentiation-dependent increase of chemerin and CMKLR1 expression was qualitatively similar to that seen for mouse adipocytes supporting a conservation of function for mice and humans.

Similar to our experiments in mouse adipocytes, we used ERK1/2 phosphorylation as a marker of CMKLR1 activation in human adipocytes. Treatment of human adipocytes with recombinant human chemerin increased (5-fold) phosphorylation of ERK1 and ERK2 MAPKs (FIG. 8F). The stimulatory effect was maximal with 1 nM chemerin and non-phosphorylated ERK2 was similar in all treatment groups. Overall, these data demonstrate that expression of chemerin and CMKLR1 is conserved and relevant in human adipose tissue.

Herein, we provide the first report that white adipose tissue expresses high levels of chemerin and its cognate receptor CMKLR1 in mice. Consistent with these in vivo data, we report the novel finding that as 3T3-L1 cells mature into adipocytes, the cells express increasing amounts of chemerin and CMKLR1 mRNA and secrete greater amounts of bioactive chemerin. Taken together, these findings demonstrate that adipocytes serve as both a primary source of chemerin secretion as well as a target for autocrine/paracrine chemerin signaling. The data derived from loss of function experiments confirm this and provide compelling evidence that a critical function of chemerin/CMKLR1 signaling is to regulate adipogenesis and metabolic homeostasis in adipocytes.

Similar to mouse, chemerin and CMKLR1 are highly expressed in human adipose. Also similar to mouse, human primary adipocytes exhibit differentiation-dependent increases in the expression of these genes as well as functional responses to exogenous chemerin treatment. Together, these findings demonstrate a conserved functional role of chemerin/CMKLR1 signaling in both mouse and human adipocyte differentiation and function.

The data provided herein provides the important recognition that adipose is unique in that this tissue expresses both high levels of chemerin and CMKLR1. Refinement of these analyses by independent consideration of the adipocyte and stromal fractions of adipose revealed that chemerin and CMKLR1 expression was enriched in the former fraction. The conclusion that adipocytes are a source of active chemerin is directly supported by our observations in 3T3-L1 mouse adipocytes. Importantly, the mature form 16 kDa, but not the precursor 18 kDa protein, was detected in the media of adipocyte-conditioned media. This demonstrates that adipocytes have the ability to both secrete and process prochemerin to the active form. Physiologically, this ability to generate mature chemerin could allow for local actions in adipose tissue without a requirement for the proteolytic enzyme secretion by other cell types, as has been shown for some neutrophil-mediated inflammatory responses.

Many adipokines act in a local autocrine/paracrine fashion to regulate adipocyte differentiation and metabolism. The estimated media concentration (390 pM) of chemerin by day 3 post-differentiation was well above the Kₘ (114 pM) for CMKLR1 activation, indicating that secretion of physiologically relevant amounts of active chemerin occurs early in adipocyte differentiation. Our finding that CMKLR1 is highly expressed in mouse adipose tissue and exhibits differentiation-dependent expression in murine and human cultured adipocytes suggests that chemerin may also have autocrine/local actions on adipocytes. Given this temporal pattern of CMKLR1 expression and chemerin secretion, the autocrine/local function of this pathway may be the regulation of signaling pathways involved in adipogenesis.

Knockdown of chemerin or CMKLR1 expression in preadipocytes severely impaired subsequent differentiation of those cells into adipocytes and reduced the expression of genes involved in glucose and lipid metabolism. A number of critical events occur within the first 72 hr of adipocyte differentiation. Twenty-four hours after inducing differentiation of 3T3-L1 cells with IBMX, dexamethasone and insulin, cells undergo a clonal expansion consisting of 1-2 rounds of cell division prior to subsequent growth arrest and commitment to the adipocyte lineage. Reinforcing cascades of early transcriptional regulators including CEBPα, CEBPγ, CEBPδ and PPARγ are also required for adipocyte differentiation during this early critical phase. The finding that chemerin and CMKLR1 knockdown largely abrogates adipocyte differentiation when initiated prior to, but not after (at day 4) the onset of differentiation, indicates that chemerin/CMKLR1 signaling is essential early in the differentiation process and may contribute to or regulate critical early events in adipogenesis. As an increase in adipocyte cell number is an important process for increasing adipose tissue mass our results indicate that chemerin and CMKLR1 have an important biological role in the formation of white adipose tissue during normal development or in pathological states such as obesity.

While knockdown of chemerin and CMKLR1 expression markedly reduces adipocyte differentiation, the highest expression of chemerin and CMKLR1 occurs in mature adipocytes. We have also observed that chemerin knockdown in the adipocyte maturation period resulted in lower expression of perilipin, GLUT4, adiponectin and leptin expression in mature adipocytes. Thus, in addition to fulfilling a vital role in adipocyte differentiation, this novel adipokine modulates metabolic pathways in mature adipocytes. The absence of chemerin expression resulted reduced basal lipolysis and IBMX-stimulated lipolysis rate. Interestingly, if adipocytes were exposed to elevated chemerin levels (1 nM) this could blunt the lipolytic response produced by the γ-adrenergic agonist isoproterenol. Catecholamine stimulation of lipolysis involves signaling through an γ-adrenergic receptor, Gs-protein, adenylyl cyclase cascade. This results in increased intracellular cAMP and activation of PKA, which in turn phosphorylates and activates HSL, a key enzyme controlling the mobilization of fatty acids from triglycerides. Previous reports indicate that nM concentrations of chemerin decrease intracellular cAMP. Thus chemerin could oppose the lipolytic action of catecholamines through the reduction of intracellular cAMP levels. IBMX, a PDE3 inhibitor, induces lipolysis by blocking the degradation of cAMP. The inability of chemerin to inhibit IBMX-stimulated lipolysis also suggests the antilipolytic mechanism lies at a point upstream of cAMP production.

While both chemerin and CMKLR1 shRNA treatment of preadipocytes impaired subsequent adipocyte differentiation, these treatments produced differences in cell morphology. Furthermore, post-differentiation knockdown of chemerin and CMKRL1 produced differential effects on gene expression and lipolysis.

Several studies have indicated that the development of insulin resistance and type II diabetes in obesity begins with local adipokine responses. In this model, increased release of adipokines (e.g. leptin, TNFα, CCL2) as well as free fatty acids from triglyceride-overloaded adipocytes stimulates macrophage infiltration and activation of a local inflammatory response. In a feed-forward system, activated macrophages release additional pro-inflammatory molecules that perpetuate the inflammatory response and impair adipocyte sensitivity to insulin. We have demonstrated that CMKLR1 is highly expressed in the stromal vascular compartment of white adipose tissue and that adipocyte-cell culture media activated human CMKLR1 and stimulated migration of CMKLR1-expressing L1.2 cells. Adipocyte-derived chemerin could act as a paracrine regulator of recruitment of CMKLR1-expressing of immune cells to white adipose tissue as part of the local inflammatory response that coincides with the development of obesity.

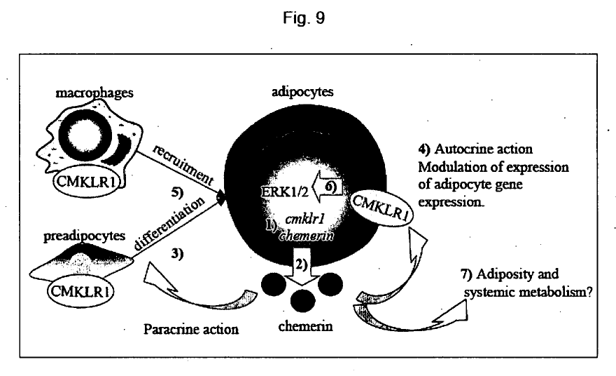

Several studies support the idea that the endocrine/systemic actions of adipokines contribute to obesity-related diseases. For example, adipokines such as resistin, TNFα, and IL6 that promote insulin resistance are elevated in obese humans or in rodent models of obesity. Other adipokines such as adiponectin have anti-diabetic and anti-inflammatory actions to decrease muscle and liver triglyceride accumulation and increase insulin sensitivity in muscle. Considering the high level of expression of chemerin in adipocytes and the increased secretion of chemerin with adipocyte maturation, the adipose depot may represent a modifiable source of chemerin secretion that changes with adipose tissue mass. Our finding that chemerin has a regulatory role in adipogenesis and adipocyte metabolism identifies the potential importance of this pathway in adipose tissue biology (FIG. 9), providing novel therapeutic approaches for the treatment of obesity, type 2 diabetes and cardiovascular disease.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

What is claimed is:

1. A method of regulating adipogenesis and/or metabolic function in adipocytes, the method comprising:
contacting mammalian adipocytic cells with an effective amount of a chemokine-like receptor 1 (CMKLR1) modulatory agent.

2. The method of claim 1, wherein the modulatory agent blocks CMKLR1 signaling.

3. The method of claim 2, wherein the modulatory agent inhibits differentiation of pre-adipocytes to adipocytes.

4. The method of claim 2, wherein the modulatory agent inhibits adipocyte metabolic function.

5. The method of claim 1, wherein the modulatory agent potentiates CMKLR1 signaling.

6. The method of claim 5, wherein the modulatory agent enhances differentiation of pre-adipocytes to adipocytes.

7. The method of claim 5, wherein the modulatory agent enhances adipocyte metabolic function.

8. The method of claim 1, wherein the adipocytic cells are present in an in vitro culture.

9. The method of claim 8, wherein said cells are human or mouse cells.

10. The method of claim 1, wherein the adipocytic cells are present in a mammalian subject.

11. The method of claim 10, wherein the subject is a mouse or human.

12. The method of claim 11, wherein the modulatory agent blocks CMKLR1 signaling and reduces fat accumulation in the subject.

13. The method of claim 1, wherein the CMKLR1 modulatory agent binds to CMKLR1.

14. The method of claim 13, wherein the CMKLR1 modulatory agent comprises a natural or altered domain derived from a natural ligand of CMKLR1.

15. The method of claim 14, wherein the natural ligand of CMKLR1 is chemerin.

16. The method of claim 13, wherein the CMKLR1 modulatory agent is an antibody or antigen binding fragment thereof.

17. The method of claim 1, wherein the CMKLR1 modulatory agent inhibits expression of CMKLR1.

18. The method of claim 17, wherein the CMKLR1 modulatory agent is a polynucleotide.

19. A method of screening for an agent that regulates adipogenesis and/or adipocyte metabolism, said method comprising:
contacting said agent with a cell expressing CMKLR1; and
evaluating whether said agent modulates CMKLR1 activity.

20. The method of claim 19, wherein the CMKLR1 activity is selected from the group consisting of: chemotaxis, activation of a signaling pathway component, activation of gene or reporter gene expression, phosphorylation of a pathway component, and expression of CMKLR1.

21. The method of claim 20, wherein said cell is genetically engineered to express CMKLR1.

22. The method of claim 20, further comprising validating said agent as regulating fat accumulation.


SEQUENCE LISTING (51 sequences)

SEQ ID NO: 1 - protein, 163 aa, human
MRRLLIPLALWLGAVGVGVAELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFSKALPRS

SEQ ID NO: 2 - protein, 9 aa, human
YFPGQFAFS

SEQ ID NO: 3 - DNA, 22 bp, mouse
agccgcttatatgtatcgctca

SEQ ID NO: 4 - DNA, 23 bp, mouse
tgccgtcataatgattctgttgg

SEQ ID NO: 5 - DNA, 23 bp, mouse
tacaggtggctctggaggagttc

SEQ ID NO: 6 - DNA, 22 bp, mouse
cttctcccgtttggtttgattg

SEQ ID NO: 7 - DNA, 20 bp, mouse
caagcaaacagccactacca

SEQ ID NO: 8 - DNA, 21 bp, mouse
tagatgccggagtcgttgtaa

SEQ ID NO: 9 - DNA, 22 bp, mouse
gagctgtttgcagacaaagttc

SEQ ID NO: 10 - DNA, 20 bp, mouse
ccctggcacatgaatcctgg

SEQ ID NO: 11 - DNA, 20 bp, mouse
tctctgtcacctggctcaac

SEQ ID NO: 12 - DNA, 20 bp, mouse
gcagtctgtgcagaaggtgt

SEQ ID NO: 13 - DNA, 21 bp, mouse
ggaggtggtgatagccggtat

SEQ ID NO: 14 - DNA, 21 bp, mouse
tgggtaatccatagagcccag

SEQ ID NO: 15 - DNA, 20 bp, mouse
ctctgtcgtccaacaccatc

SEQ ID NO: 16 - DNA, 20 bp, mouse
ctcgttcttcttgggctttc

SEQ ID NO: 17 - DNA, 20 bp, mouse
actcttgccacacaggctct

SEQ ID NO: 18 - DNA, 20 bp, mouse
aatggagactgatgcgctct

SEQ ID NO: 19 - DNA, 20 bp, mouse
gcttggttcaactggagagc

SEQ ID NO: 20 - DNA, 20 bp, mouse
gcctagtgccttctggtctg

SEQ ID NO: 21 - DNA, 23 bp, mouse
tagtccttcctaccccaatttcc

SEQ ID NO: 22 - DNA, 21 bp, mouse
ttggtccttagccactccttc

SEQ ID NO: 23 - DNA, 20 bp, mouse
cctgtaccctggagaggtgt

SEQ ID NO: 24 - DNA, 20 bp, mouse
cggatgactgtgagatttgg

SEQ ID NO: 25 - DNA, 19 bp, mouse
gagacccctgtgtcggttc

SEQ ID NO: 26 - DNA, 22 bp, mouse
ctgcgtgtgtgaaatgtcattg

SEQ ID NO: 27 - DNA, 20 bp, mouse
gtggtgcagctcatcaagaa

SEQ ID NO: 28 - DNA, 20 bp, mouse
gccatgacctttacctggaa

SEQ ID NO: 29 - DNA, 20 bp, mouse
acactctccggaacaccatc

SEQ ID NO: 30 - DNA, 20 bp, mouse
ccctccctttggtagaggag

SEQ ID NO: 31 - DNA, 20 bp, mouse
tcgctgatgcactgcctatg

SEQ ID NO: 32 - DNA, 21 bp, mouse
gagaggtccacagagctgaat

SEQ ID NO: 33 - DNA, 20 bp, mouse
ctggacctaccggcatgttc

SEQ ID NO: 34 - DNA, 19 bp, mouse
gtcatcccgctcccaacac

SEQ ID NO: 35 - DNA, 23 bp, mouse
ccctcacactcagatcatcttct

SEQ ID NO: 36 - DNA, 19 bp, mouse
gctacgacgtgggctacag

SEQ ID NO: 37 - DNA, 21 bp, human
tggaagaaacccgagtgcaaa

SEQ ID NO: 38 - DNA, 21 bp, human
agaacttgggtctctatgggg

SEQ ID NO: 39 - DNA, 24 bp, human
atggactaccactgggttttcggg

SEQ ID NO: 40 - DNA, 25 bp, human
gaagacgagagatggggaactcaag

SEQ ID NO: 41 - DNA, 20 bp, human
ttcatctgcactgccaagac

SEQ ID NO: 42 - DNA, 20 bp, human
tcgagttgtccacagtcagc

SEQ ID NO: 43 - DNA, 20 bp, human
ggctttggccctatcttttc

SEQ ID NO: 44 - DNA, 20 bp, human
ccaaaccggtgactttctgt

SEQ ID NO: 45 - DNA, 20 bp, human
gagcccaagtttgagtttgc

SEQ ID NO: 46 - DNA, 20 bp, human
ctgtgaggactcagggtggt

SEQ ID NO: 47 - DNA, 49 bp, mouse
accggatagtccactgcccaattccgaagaattgggcagtggactatcc

SEQ ID NO: 48 - DNA, 50 bp, mouse
aaaaggatagtccactgcccaattcttcggaattgggcagtggactatcc

SEQ ID NO: 49 - DNA, 50 bp, mouse
caccggaagataacctgcttcaacacgaatgttgaagcaggttatcttcc

SEQ ID NO: 50 - DNA, 50 bp, mouse
aaaaggaagataacctgcttcaacattcgtgttgaagcaggttatcttcc

SEQ ID NO: 51 - protein, 15 aa, mouse
CLAFQEIGVDRAEEV